(19) United States
(12) Patent Application Publication
(10) Pub. No.: US 2016/0096073 A1
(43) Pub. Date: Apr. 7, 2016

(54) GAME-BASED METHOD AND SYSTEM FOR PHYSICAL REHABILITATION

(71) Applicant: Umm Al-Qura University, Makkah (SA)

(72) Inventors: Mohamed Abdur RAHMAN, Makkah (SA); Faizan Ur Rehman, Makkah (SA); Saleh Basalamah, Makkah (SA)

(73) Assignee: Umm Al-Qura University, Makkah (SA)

(21) Appl. No.: 14/877,209

(22) Filed: Oct. 7, 2015

Related U.S. Application Data
(60) Provisional application No. 62/060,981, filed on Oct. 7, 2014.
(60) Provisional application No. 62/141,719, filed on Apr. 1, 2015.

(51) Int. Cl. A63B 24/00; A63F 13/798; A61B 5/11; A63F 13/428; A63F 13/211; G06F 3/01; A63F 13/67; A63F 13/23

(52) U.S. Cl. CPC A63B 24/0062; A63F 13/67; A63F 13/798; A63F 13/23; A63F 13/428; A63F 13/211; G06F 3/011; A61B 5/11; A63B 2024/0068; A63B 2024/0071; A63B 2220/803; A63B 2220/30

(57) ABSTRACT

A game-based method for physical rehabilitation that includes authenticating a user with authentication information input through a communication interface, identifying therapeutic movements by referencing a look-up table stored in a memory, the therapeutic movements being prescribed for the user or associated with a diagnosis of a predetermined physical condition, obtaining a user rehabilitation status stored in the memory, generating a game based on the therapeutic movements and the user status wherein the game includes controlling browsing of a map by detecting the therapeutic movements performed by the user, providing the game to the user, receiving a data stream from a motion-sensing device that monitors an actual movement of the user in correspondence to the therapeutic movements, analyzing the data stream to calculate information metrics that are associated with the correspondence of the actual movements to the therapeutic movements, and updating the user rehabilitation status based on the information metrics.

### CROSS REFERENCE

This application claims the benefit of priority from U.S. Provisional Application No. 62/060,981 filed Oct. 7, 2014 and from U.S. Provisional Application No. 62/141,719 filed Apr. 1, 2015, both of which are herein incorporated by reference in their entirety.

### BACKGROUND

Hemiplegia is a disability that renders half of a patient's hand immovable. Therapy includes exercises to move the affected joints and muscles. The therapeutic exercises are prescribed to patients by medical professionals. In order to provide quality of service, therapists need to know the certain kinematic metrics that require the use of certain devices that need to be brought in proximity of the patient's hand. Therapy in the home is more flexible and more convenient for the patient by allowing more frequent repetition of therapy exercises.

Accordingly, what is needed as recognized by the present inventor is a system that uses noninvasive technologies to track and monitor joints. In addition, therapeutic exercises need to be simple, entertaining and may be performed and monitored outside of a clinical setting.

The foregoing “background” description is for the purpose of generally presenting the context of the disclosure. Work of the inventor, to the extent it is described in this background section, as well as aspects of the description which may not otherwise qualify as prior art at the time of filing, are neither expressly or impliedly admitted as prior art against the present invention. The foregoing paragraphs have been provided by way of general introduction, and are not intended to limit the scope of the following claims. The described embodiments, together with further advantages, will be best understood by reference to the following detailed description taken in conjunction with the accompanying drawings.

### SUMMARY

A game-based method for physical rehabilitation is provided that authenticates via processing circuitry, a user with authentication information input through a communication interface, identifies therapeutic movements by referencing a look-up table stored in a memory, the therapeutic movements being prescribed for the user or associated with a diagnosis of a predetermined physical condition, obtains a user rehabilitation status stored in the memory, generates a game based on the therapeutic movements and the user status wherein the game includes controlling browsing of a map by detecting the therapeutic movements performed by the user, provides the game to the user, receives a data stream from a motion-sensing device that monitors an actual movement of the user in correspondence to the therapeutic movements, analyzes the data stream to calculate information metrics that are associated with the correspondence of the actual movements to the therapeutic movements, and updates the user rehabilitation status based on the information metrics.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 is a schematic representation of the system according to one example;

FIG. 2 is a schematic of a sample therapy environment according to one example;

FIG. 3 is a schematic that shows anatomy of joints in a hand according to one example;

FIG. 4A is a schematic that shows primitive motion detected by the system according to one example;

FIG. 4B is a schematic that shows primitive motion detected by the system according to one example;

FIG. 5 is a schematic that shows a primitive motion of squeezing and enlarging a palm surface area according to one example;

FIG. 6 is a block diagram representation of the system according to one example;

FIG. 7 is a block diagram representation of the system using a serious game according to one example;

FIG. 8 is a flow chart to store a therapeutic session according to one example;

FIG. 9 is a flow chart for a motion analyzer according to one example;

FIG. 10 is a flow chart that shows an algorithm used by a reporting engine according to one example;

FIG. 11 is an exemplary user interface to choose joints and movements according to one example;

FIG. 12 is a schematic that shows a hand movement according to one example;

FIG. 13 is a schematic that shows complex and compound therapies according to one example;

FIG. 14 is a schematic that shows complex and compound therapies according to one example;

FIG. 15 is a schematic that shows complex and compound therapies according to one example;

FIG. 16 is an exemplary flow chart that shows the operation of the system according to one example;

FIG. 17 is a flow chart that shows the operation of the system according to one example;

FIG. 18 is a flow chart that shows an algorithm to generate a serious game according to one example;

FIG. 19 is a schematic that shows an exemplary 3D live view of a therapeutic exercise according to one example;

FIG. 20 is a schematic that shows the range of motion of a joint according to one example;

FIG. 21 is an image that shows an exemplary system setup according to one example;

FIG. 22 is an exemplary web based user interface according to one example;

FIG. 23 is a chart that shows exemplary traces obtained from the inverse kinematic analyzer according to one example;

FIG. 24 is an exemplary block diagram of a server according to one example;

FIG. 25 is an exemplary block diagram of a data processing system according to one example; and

FIG. 26 is an exemplary block diagram of a central processing unit according to one example.

### DETAILED DESCRIPTION

Referring now to the drawings, wherein like reference numerals designate identical or corresponding parts throughout several views, the following description relates to a system and associated methodology for detecting, tracking, and visualization of physical therapy data.

The present disclosure relates to using a motion-sensing device such as LEAP1 to detect, recognize and track the rotational and angular movements of different joints of the body. Kinematic and therapeutic data are then extracted from these movements. The system and associated methodology calculate a range of motion (ROM) from each therapy session. The range of motion is then displayed to the user in real time. A detailed analysis may also be displayed to the user. The analysis may be plotted in a 3D environment. The system of the present disclosure has the advantage of being non-invasive, as the patient does not need to wear any external devices. Complex measurement devices are bulky and restrict movements for even the normal patient. This is a non-invasive device and hence can be used with disabled hemiplegic patients of any level.

The method of the present disclosure may also incorporate a 3D webGL-based serious game environment where the live therapeutic movement of the patient and a therapist is synchronized between a physical and a 3D environment. Since the framework is web-based, the user just needs the motion-sensing device to be connected to an electronic device. The electronic device connects via a network to a server. The user may then use a web browser to use the system of the present disclosure. In addition, the system and associated methodology of the present disclosure detects, recognizes and visualizes primitive hand gestures and then use them to define high-level and complex therapies that combine the primitive hand gestures.

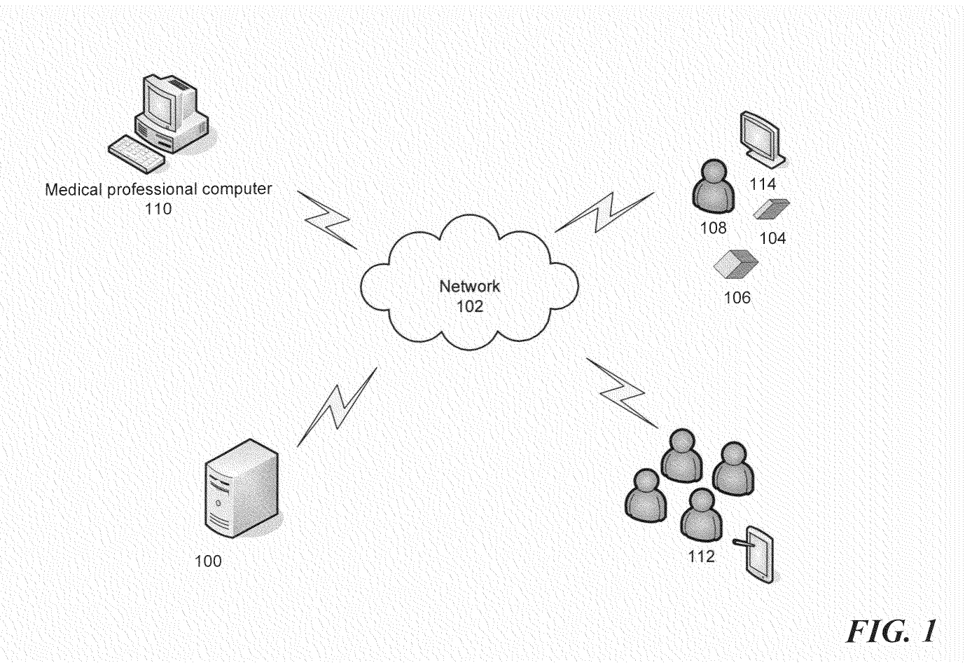

FIG. 1 is a schematic representation of the system according to one example. FIG. 1 shows an exemplary system of motion-sensing devices 104, 106 connected within a system having the server 100, a network 102, a medical professional computer 110, an electronic device 114 and a community of interest 112. The medical professional computer 110 may connect to the server 100 through the network 102. A patient 108 may use one or more motion-sensing devices. The motion-sensing devices 104, 106 capture the patient's 108 movements during a therapy session. The motion-sensing devices 104, 106 may include communication circuitry to transmit the recorded user motions through the network 102 to the server 100.

The network 102 is any network that allows the motion-sensing device and the medical professional computer 110 to communicate information with each other such as Wide Area Network, Local Area Network or the Internet. The medical professional computer 110 represents one or more medical professional computers that could be located in a doctor's office, hospital or other medical facility or health facility where they are used in the treating of patients as well as the review of patient records. The patient 108 may also use a personal computer to connect to the server 100 through the network 102 to view personal medical records and therapeutic exercises. In one embodiment, the personal computer may be connected with the motion sensing devices 104 and 106.

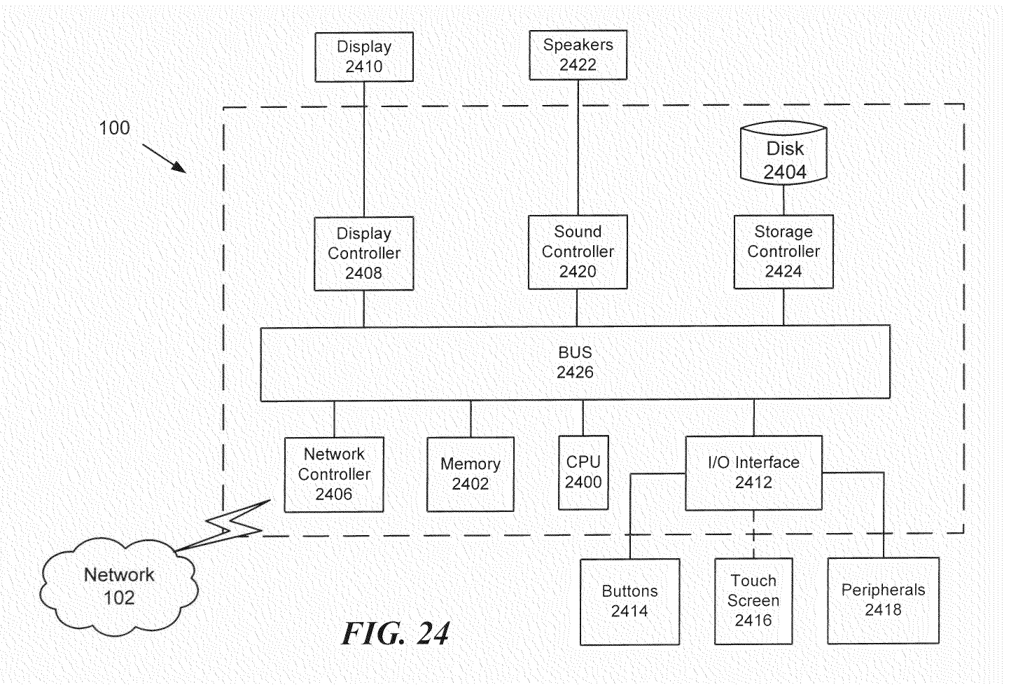

The server 100 may be or include any database configured to store and/or provide access to information, such as an electronic database, a computer and/or computerized server, database server or any network host configured to store data. Further, the server 100 may include or be a part of a distributed network or cloud computing environment. As shown in FIG. 24, the server 100 may include a CPU 2400 and a memory 2402.

The motion-sensing device 104 may be any device configured to detect a 3D movement. The motion-sensing device may be based on different types of technologies. For example, the motion-sensing device may use accelerometers to detect orientation and acceleration. The motion-sensing device may use infrared structured light. For example, the motion-sensing device may be a LEAP or KINECT device. The LEAP device is a 3D-sensor device that captures all the motion of hands and fingers at a rate of 60 frames per second. The KINECT device captures the 3D movement data of all the joints in the body. The KINECT device may capture motions from 20 joints of a human body at a rate of 30 frames per second. In one embodiment, the motion-sensing device may also receive data from one or more sensors that senses motions of the user.

The user may use any electronic device connected to the motion-sensing device to visualize interfaces. The electronic device 114 may be a computer, a laptop, a smartphone, a tablet, a television, or the like. The electronic device may include a computer monitor, television, or projector that can be used to view the output from the system. The electronic device 114 may be connected to the motion-sensing device 104 using a wired or wireless connection. In one embodiment, when the connection to the server 100 is not available, the motion-sensing device may store the captured data. Once a connection becomes available, the motion-sensing device may upload the captured data to the server 100 via the network 102. In one embodiment, the server 100 may poll the motion-sensing device, at predetermined instances, to check whether updated data is available. In response, to determining that new data is available the data are uploaded to the server 100 using the network 102. The data may then be processed in the server 100. The user may then download the data to the electronic device 114.

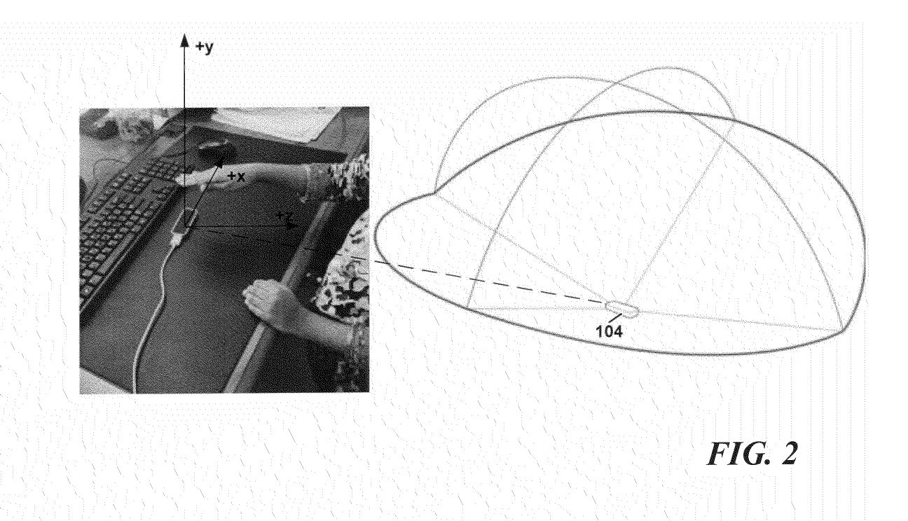

FIG. 2 is a schematic of a sample therapy environment according to one example. FIG. 2 shows the motion-sensing device 104, a user hand, and a 3D sensing range. In one embodiment, the motion-sensing device may be that manufactured by LEAP MOTION as shown in FIG. 2. The motion-sensing device may use one or more camera to capture the user hand movement. The 3D sensing range depends on the motion-sensing device used. For example, for the LEAP device, the 3D sensing range is a roughly hemispherical area, to a distance of about 1 meter.

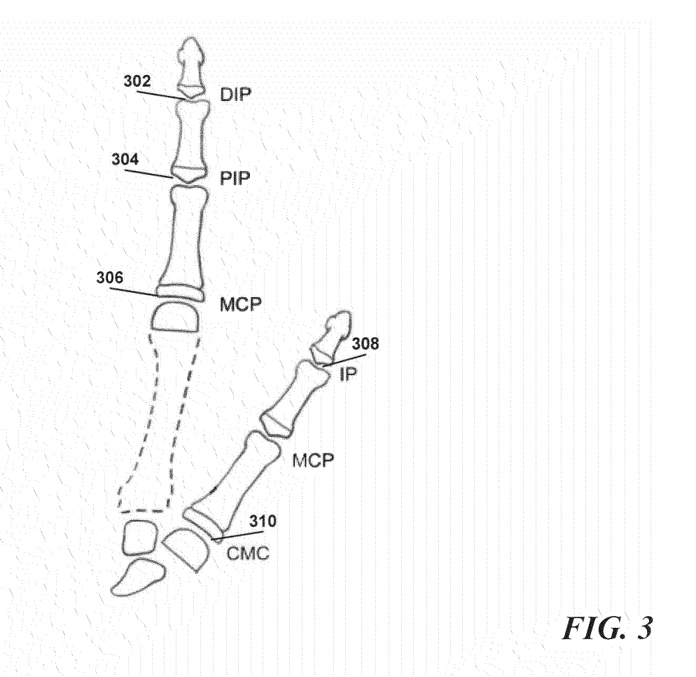

FIG. 3 is a schematic that shows anatomy of joints in a hand according to one example. FIG. 3 shows exemplary joints that can be monitored and tracked using the system of the present disclosure. Joints in the index include a Distal inter-phalangeal (DIP) joint 302, a Proximal Inter-Phalangeal (PIP) joint 304, Meta-Carpo-Phalangeal (MCP) joint 306. The joints in the thumb include an interphalangeal joint 308, the MCP joint 306, a carpometacarpal joint 310.

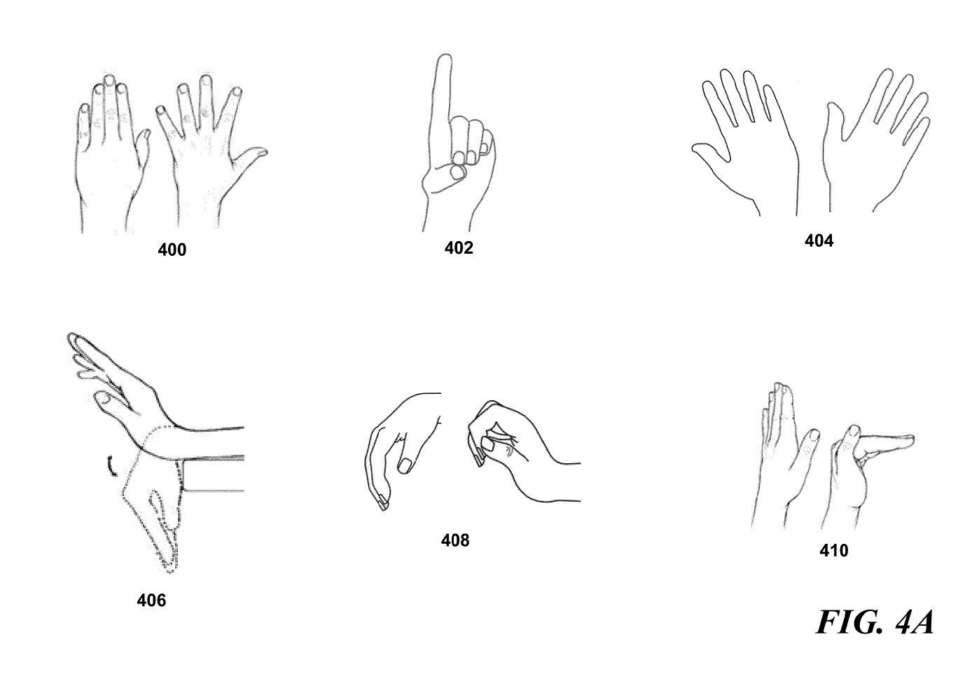

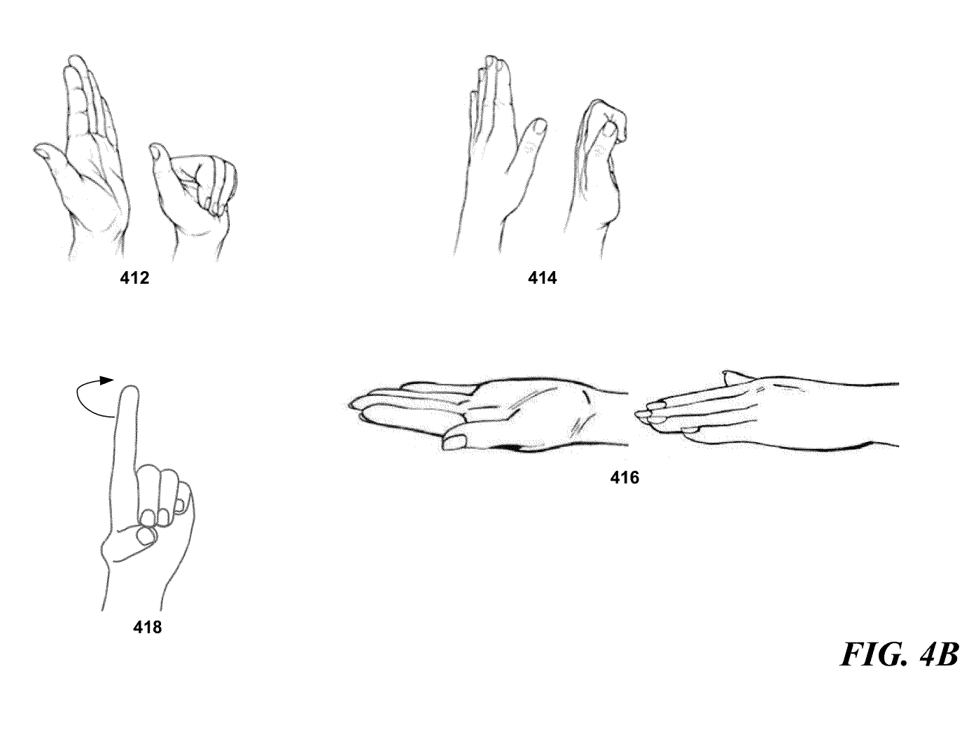

FIGS. 4A and 4B are schematics that show primitive motion detected by the system according to one example. The system and associated methodology may detect and plot in real time primitive motions including but not limited to abduction/adduction of all fingers and thumb 400, abduction/adduction of a single finger 402, radial/ulnar deviation around wrist joint 404, hyper-flexion/hyper-extension around wrist joint 406, flexion/extension around wrist joint 408, flexion/extension around MCP joints 410, flexion/extension around PIP joints 412, Flexion/extension around DIP 414, forearm pronation/supination 416, and circumduction 418. The circumduction movement 418 shows the primitive motion of a circumduction action around the index finger.

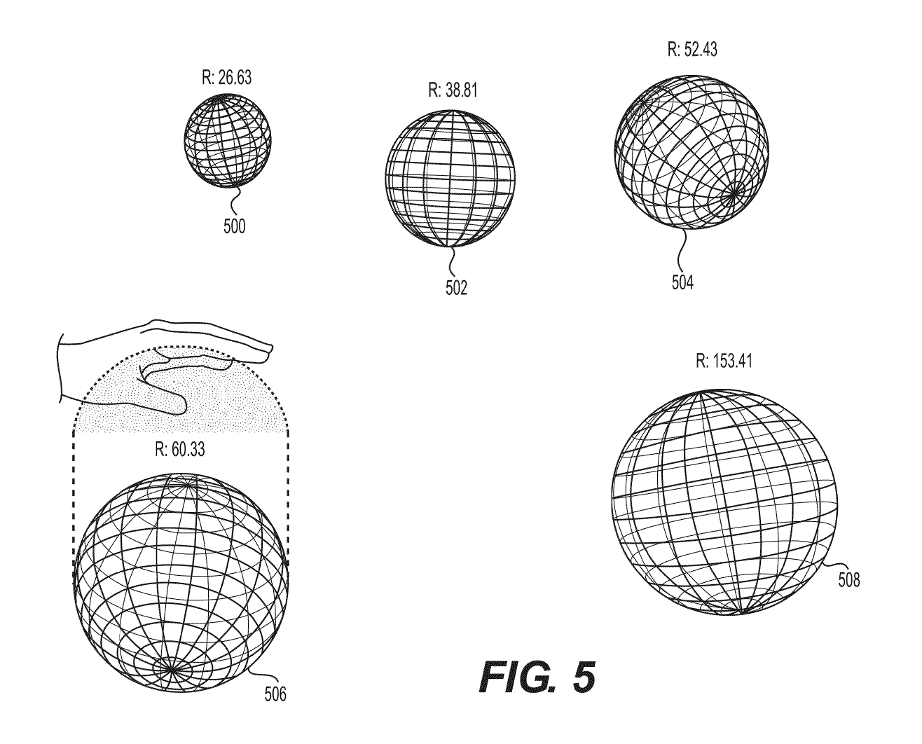

FIG. 5 is a schematic that shows a primitive motion of squeezing and enlarging a palm surface area according to one example. Hand therapy to infer the capability of squeezing or enlarging the palm surface area may be studied by measuring the radius of imaginary sphere from data collected by the motion-sensing device. A first sphere 500 has a radius of 26.63 cm and represents the average sphere radius while doing a pinch operation or holding a pen. A second sphere 502 has a radius of 38.81 cm and represents the movement needed to hold a coffee mug at its handle. A third sphere 504 has a radius of 52.43 cm and represents the movement needed to hold an iPhone 5 as an example. A fourth sphere 506 has a radius of 60.33 cm and represents the average movement to hold a coffee mug from its top. A fifth sphere 508 has a radius of 153.41 cm and represents the average movement of holding a coffee mug from its bottom in which the hand palm is in a pronation position.

As discussed above, each joint has a number of movements associated with it. Some movements are angular for example flexion/extension of the elbow that takes place when the wrist is brought near the shoulder or moved away from it. Let J be the set of joints being tracked:

J={j₁,j₂,j₃, . . . ,jₘ} (1)

For example, J can be j₁=finger MCP, j₂=right shoulder, j₃=left shoulder.
At any given temporal dimension, the joint has a particular state and at that state the joint produce one or more movements related to that state. A set of states may be defined as follows:

S={s₁,s₂,s₃, . . . ,sₘ} (2)

For example, S may be s₁=flexion, s₂=extension, s₃=abduction, s₄=adduction.
A primitive therapeutic context Pᵢ may be defined as a set of ordered pairs of joints and their respective states as following:

Pᵢ={jₘ,sₙ>} (3)

For example, P1 may represent the primitive therapeutic contexts for wrist flexion and P2 may represent the primitive therapeutic context for wrist extension

P₁={j₁,s₁} (4)

P₂={j₁,s₂} (5)

A complete therapeutic context T is defined as a series of primitive therapeutic contexts P₁ . . . Pₙ. As an example the above two primitive therapeutic contexts can be serially combined into a complete therapeutic context T depicting the wrist bend therapy as follows:

T={P₁,P₂} (6)

where P₁ is wrist-flexion and P₂ is wrist extension.

A high level therapy may be composed of a number of sub-therapeutic. For example, “walking” therapeutic exercise may be broken down into three separate sub-therapeutic actions around three different joints that need to be monitored. The three joints and their associated movements are: flexion/extension of hip joint, flexion/extension of knee joint and dorsiflexion/planter of ankle joint. The system then tracks movement or motion using the modeling described above.

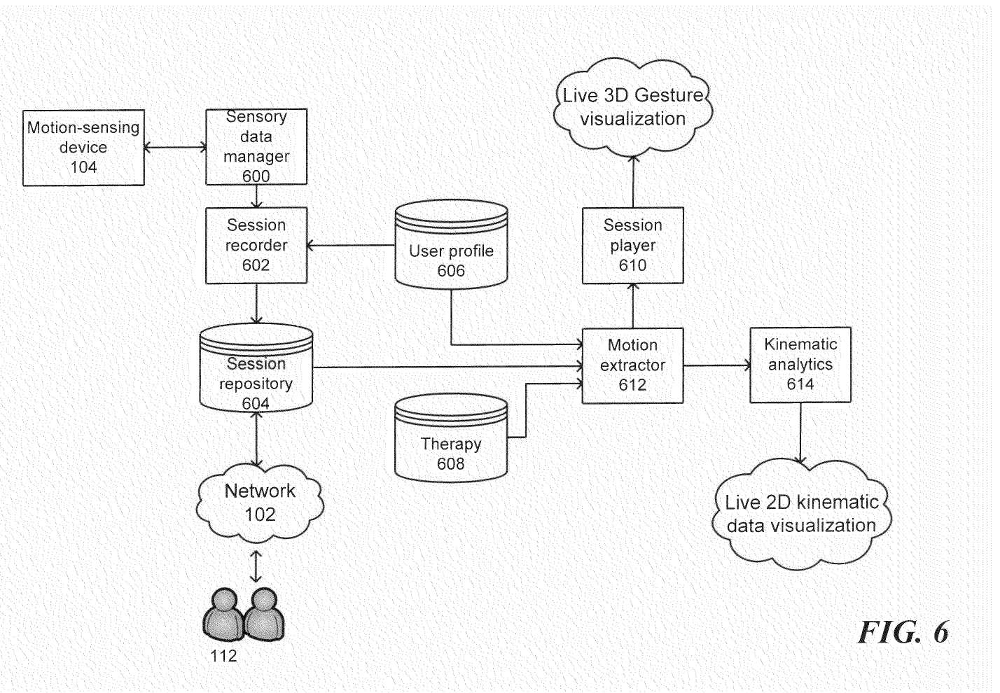

FIG. 6 is a block diagram representation of the system according to one example. FIG. 6 shows a high-level framework in which motion data is collected from a multi-sensory environment. The multi-sensory environment may include one or more motion-sensing devices 104, 106. The system may be used by different types of users. The user may be the patient, a therapist or a caregiver such as parents. Depending on the user type, a different set of services and user interfaces may be available. Two or more visualization interfaces may be used by the system. A first interface may show a therapeutic activity in real time. The system may use a 3D web interface to show the therapeutic activity. In one embodiment, the therapeutic activity may be in the form of a serious game. A second interface may show an analytical output where live plotting of different quality of performance metrics is shown. Both the therapist and the patient may use the system to either record or playback therapy sessions. The therapy session may be replayed by either playing a pre-encoded video or by re-rendering the stored data points using in browser 3D rendering techniques such as WebGL. The re-rendering may be done on the server 100 and then sent to the medical professional computer 110 and to the electronic device 114, or the re-rendering may be done on the medical professional computer 110 and on the electronic device. A session can be controlled by a menu driven interface as well as a speech-based interface. The server 100 using the CPU 2400 may use voice recognition technique to detect the user input. The ability to control the system using the speech-based interface is useful when the patient cannot use his hands to make the selection.

In one embodiment, the user may need to be authenticated before starting using the system. The authentication can be performed by a variety of methods such as voice recognition via a microphone or fingerprint recognition via a fingerprint sensor. The fingerprint is verified using the fingerprint verification circuitry by comparing the fingerprint with the fingerprint stored in a user profile. In other example, the user may be authenticated by entering a pin code. At the beginning of each session, the patient 108 may indicate what devices are available to him. The patient may use the speech-based interface or the menu driven interface to choose the available devices.

The system may include a sensory data manager 600. The sensory data manager 600 processes raw data from the motion-sensing device to extract joint data. In one embodiment, the raw data frames are in a JavaScript Object Notation (JSON) format. The extracted joint data contains the locations of joints as observed at a predetermined rate. For example, the locations of hand joints may be observed 60 times per second using the LEAP device. The predetermined rate may depend on the maximum acquisition rate of the motion-sensing device 104 type. In other embodiments, the predetermined rate may be in function of the required resolution.

The system may also include a session recorder 602. The session recorder may record the therapy session. The recorded therapy session may be saved to a session repository 604. The recorded therapy session may also be used by a motion extractor 612 to provide a live-view in a 3D environment. The motion extractor 612 may also provide plotting of the different quality of performance metrics in real time. The user may choose to record the therapy session or not. For example, the user may choose not to save the therapy session when an error has occurred. For example, the patient may start the session then stop for a particular reason. The user may also choose which joints need to be tracked. The selected joints are then displayed in real time.

The session repository database 604 stores the session data. The session data may also be stored in a cloud based secondary storage. The session data may be then accessed and played later by the user. The community of interest (COI) 112 may also access the session data using the network 102. The community of interest 112 may include caregivers. The COI 112 may include patient's parents, family members, relatives, friends, medical professional or the like. A medical professional is any member of a health-related field having a professional status or membership as would be understood by one of ordinary skill in the art. The COI may be authenticated before allowing access. Access to the session data may be limited depending on the medical professional level, experience, special privileges or seniority. In other words, the access to the session record may be restricted by the CPU 2400. For example, a nurse may display the session data but cannot delete or update the session data. In another example, relatives may display the patient status but may not be able to display detailed information about the patient health. The session data may also be added to the patient online electronic health record for sharing purposes. The system may also include a user profile database 606 and a therapy database 608.

The user profile database 606 stores electronic medical records (EMR). The user profile database 606 stores detailed information about the patient, the therapist and the caregiver. The patient identification information may include one or more of, but not limited to, a name, a photo, a date of birth, a weight, a height, a gender, a skin color, a hair color, a next of kin, a fingerprint, an address, an emergency contact number and an identification number.

The user profile database 606 may also store a patient medical record. The patient medical record can include one or more of, but not limited to, a blood type, a vaccination list, an allergy list, a past surgeries list, insurance company information, a genetic diseases list, an immunization list, a family medical history and a prescribed medicament list. In addition, the user profile database stores disability information. The disability information may include one or more of, but not limited to, type of disability, therapist name, past history of therapy, recorded sessions, and improvement parameters.

The therapy database 608 stores details about the disability type, a therapy identification, therapy type, types of motions involved in each therapy type, joints and muscles to be tracked in each motion type, metrics that store those joint and muscle values, normal range of each of the motions and metrics, improvement metrics for each disability type, and the like. The therapy database 608 may also include information about specific clinical syndrome.

The motion extractor 612 may combine session data with user preferences from the user profile database 606 and data from the therapy database 608 and provides the output to a session player 610 and to a kinematic analytics module 614.

The kinematic analytics module 614 employs analytics and algorithms to provide live visualization of different quality of improvement metrics for each selected joints.

The session player 610 manages the movement of joints in the interface. For example, the session player 610 manages the movement of the physical hand in the 3D visualization interface.

As mentioned above, the second interface shows live 2D kinematic data. The second interface shows the joint positions, range of motion around each joint, speed and other metrics over the course of the therapy session. The graphs may be plotted in real time during the session. In an embodiment, the graphs may be plotted after a session is completed. The visualization interface is used to start and end the therapy session.

In one embodiment, the medical professional may monitor the therapy session in real time. The medical professional may then provide live feedback to the patient. For example, when the patient receives a new prescribed therapeutic sequence of exercises, the medical professional may monitor the patient and provides a live feedback to the patient via the network 102. For example, the medical professional may correct the patient moves.

In one embodiment, audio to encourage the patient 104 may be generated by the server 100. For example, when the patient 104 performance is better than a predetermined criterion a prerecorded sentence may be played. The predetermined criterion may be a performance better than the patient average performance. In other embodiments, the predetermined criterion may be a goal set by the medical professional. The CPU 2400 may generate a target for a therapeutic session based on the user past performance, other patients' performances, and the user profile. This is done by comparing patient's performance against reference data stored in the therapy database 608. Once the patient achieves the goal, the audio is generated. For example, the CPU 2400 may analyze past data, to determine that the patient is showing an improvement of 1% after each therapeutic session. The CPU 2400 may obtain the current state of the joint from the user health record and may calculate a target state with the 1% improvement. Once the target is reached, which implies that the patient achieved the 1% improvement. The audio may be played. The audio generated may be based on the patient's age. For example, for a young girl the audio may be played using the voice of a Disney princess. In other embodiments, other encouragement methods may be used. For example, once a child completes the therapeutic session, the system may display the child favorite songs or the child favorite cartoon.

The therapeutic sequence of exercises that the patient follows and executes may be delivered to the user in a plurality of ways. For example, the therapeutic exercises may be prerecorded on a DVD. The exercises may be demonstrated using an avatar on the screen. The system may use online virtual game such as Second Life to display the movements of the therapist. The virtual online environment may have a similar design than the rehabilitation center where the patient goes. The patient may log in to the virtual online environment to view or download a practice session.

The system may also include an authoring interface. The authoring interface may be used by the medical professional to design a new therapy. The new therapy may include new exercises or a new therapeutic exercise sequence. The new therapy may be stored in the therapy database 608. The new therapy is then available to other medical professionals. The medical professional may choose to create a new therapy or modify an existing therapy. The system may associate a score with each therapy. The score is a function of past patients improvement using the therapy.

In one embodiment, the therapeutic exercises may be presented to the user using a serious game format. The serious game may be a health GIS game. The hand gestures are then captured while the patient is playing the game. The game may be played by multiplayer. The other player may be a healthy individual, which may encourage kids to play while performing their required therapeutic exercises.

In one embodiment, the health GIS game may consist of browsing a map. The patient 108 may be presented with a map on the display of the electronic device. The user gesture may be captured while browsing the map. The game may include browsing the map in order to find virtual checkpoints. The game may be in a 2D or 3D format. The serious game may be designed to implement a therapeutic session of exercises consisting of six movements for the forearm and two joints. The serious game consists of browsing a map by going left (radial deviation), right (ulnar deviation), zoom in (wrist flexion), zoom out (wrist extension/hyperextension), and circling around an airplane (pronation/supination). The serious game virtual background may be based on the patient age. For example, a cartoon like map may be used for a child.

Table 1 shows exemplary mapping of joint movement with the map movement. Table 1 shows one exemplary configuration. Other configurations may be used based on the patient's health condition. The other configurations may be stored in the server 100. For example, the therapist may indicate the patient health condition and the processing circuitry may determine a suitable configuration for the patient. For example, when the patient is missing limbs or fingers the suitable configuration may not include a map movement that requires the movement of the missing limbs. For example, the abduction/adduction movement 400 to move up in the map may be replaced by a radial deviation in the right hand when the patient is missing fingers. The map may correspond to the map where the patient resides.

TABLE 1
Mapping of joint movement with the map movement

| | | | Range of | | |
|---|---|---|---|---|---|
| | | | Motion | | |
| Hand/Forearm | | Map | (Normal | | |
| gesture | Therapy | movement | range) | Device | Body part |
| 404 | Radial deviation | Go left | 0-20 | Leap | Wrist |
| 404 | Ulnar deviation | Go Right | 0-30 | Leap | Wrist |
| 410 | Hyper Flexion | Zoom Out | 0-60 | Leap | Wrist, |
| | | | | | Fingers |
| 410 | Hyper Extension | Zoom in | 0-90 | Leap | Wrist, |
| | | | | | Fingers |
| 400 | Abduction/Adduction | Move Up | 0-20 | Leap | Fingers |
| 412 | Flexion of MCP joints | Move Up, | Based on the | Leap, | Palm, |
| | of fingers, elbow | down, left, | combination | Kinect | elbow, |
| | flexion/extension and | right | of movements | | shoulder |
| | shoulder | | of Palm, | | |
| | flexion/extension | | elbow and | | |
| | | | shoulder | | |
| Required both | Same as above for both | Zoom in | Based on the | Leap, | Palm, |
| hands | hands | and zoom | distance | Kinect | elbow, |
| | | out | between two | | and |
| | | | hands | | shoulder |
| | | | | | for both |
| | | | | | hands |

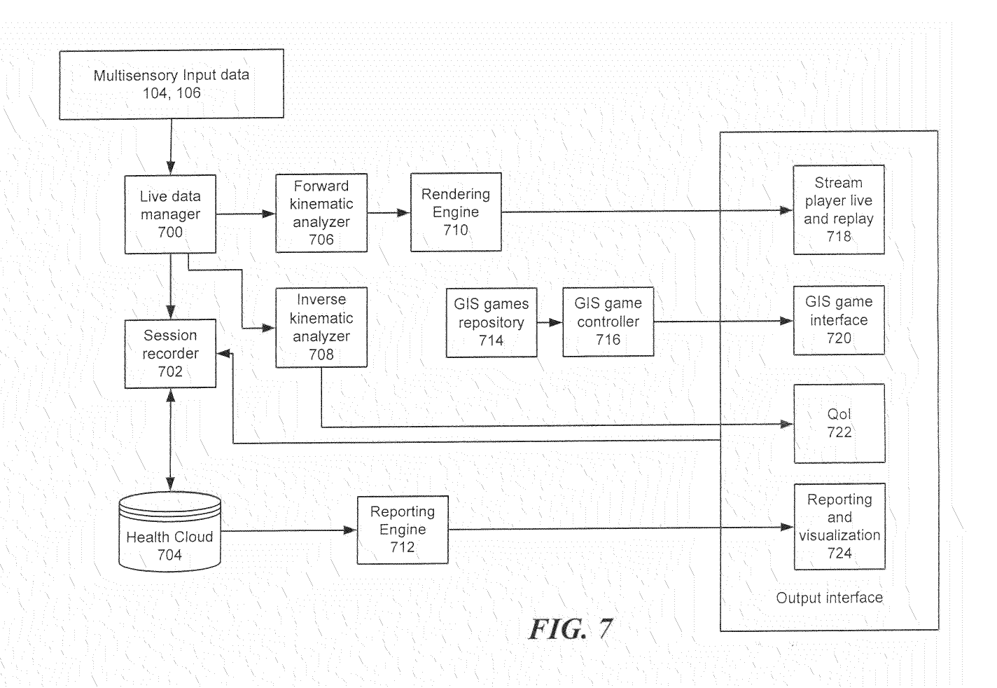

FIG. 7 is a block diagram representation of the system using a HealthGIS game according to one example. A live data manager 700 collects 3D raw data stream from the motion-sensing devices 104, 106 and forwards the 3D raw data stream to an inverse kinematics analyzer 708 for online analysis. A rendering engine 710 detects the display type of the electronic device 114 of the patient. The rendering engine 710 displays the data on the screen in the proper format corresponding to the detected display type. The rendering engine 710 receives the input stream from a forward kinematic analyzer 706. The KINECT stream may be rendered as an animated skeleton. The LEAP stream may be shown as a box figure.

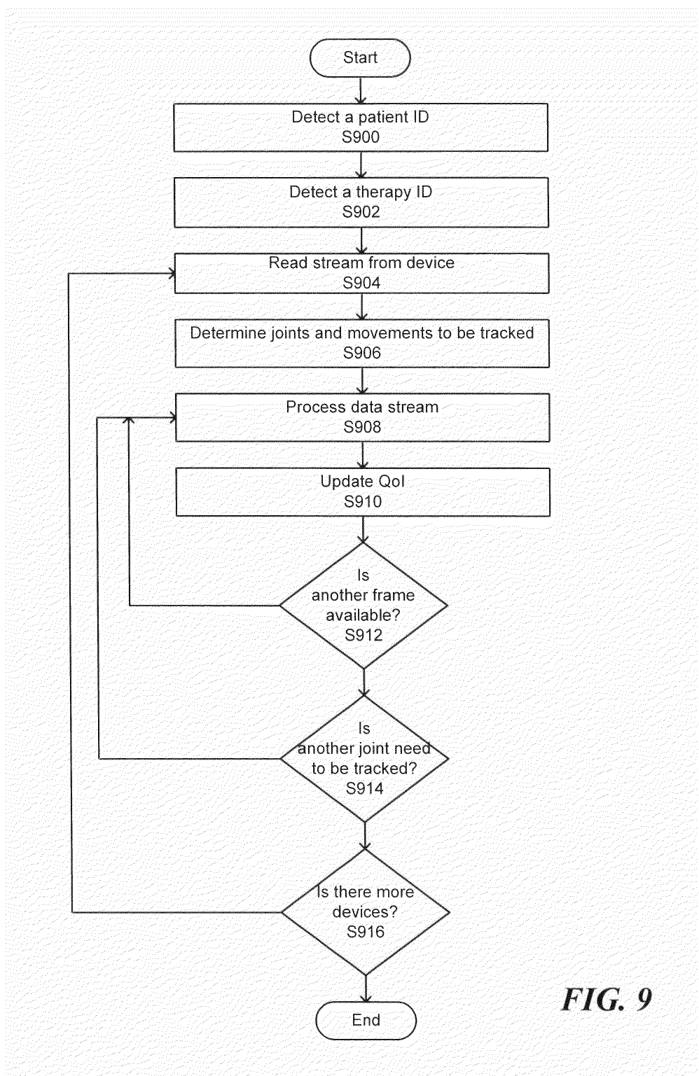

An inverse kinematics analyzer 708 processes the data and detects the state of the joints and motions in the live stream. The system also provides information to the analyzer regarding the joints that need to be tracked. The analyzer calls the function required to parse the stream. The output is forwarded to the appropriate window in the user interface to inform the user about information to improve the quality of improvement (QoI). The algorithm for the LEAP and KINECT motion analyzer is shown in flow chart shown in FIG. 9.

The user interface may include a quality of improvement display window 722. The quality of improvement window may display the name of the motion of the joints being tracked, for example, supination or pronation of the forearm. The motion name is received from the inverse kinematics analyzer 708.

The session recorder 702 may record the data stream. The data stream may be stored in a memory of the electronic device of the user. The data stream may also be uploaded to a health cloud 707. The data stream may be stored in a JavaScript object Notation (JSON) or a Bio Vision Hierarchy (BVH) format.

In one embodiment, the user may control the session recorder using the system interface. The user may click a button to start, stop or pause the recording. The user has the ability to start the recoding when he is ready to perform the therapeutic exercises. The user may pause the recording in the case of interference from clothes or objects in the environment. In one embodiment, the live stream display may continue even when the recording is paused. The user can hence get a visual clue when the interference is removed and can continue with the recording. As discussed above, the system may use voice control to accept the user input. The user may speak a single command. Then, the server may match the single command with a corresponding action. The server 100 may then execute the voice command. In one embodiment, the single command may also be used to authenticate and identify the user by comparing the voice command with stored speech model as would be understood by one of ordinary skill in the art.

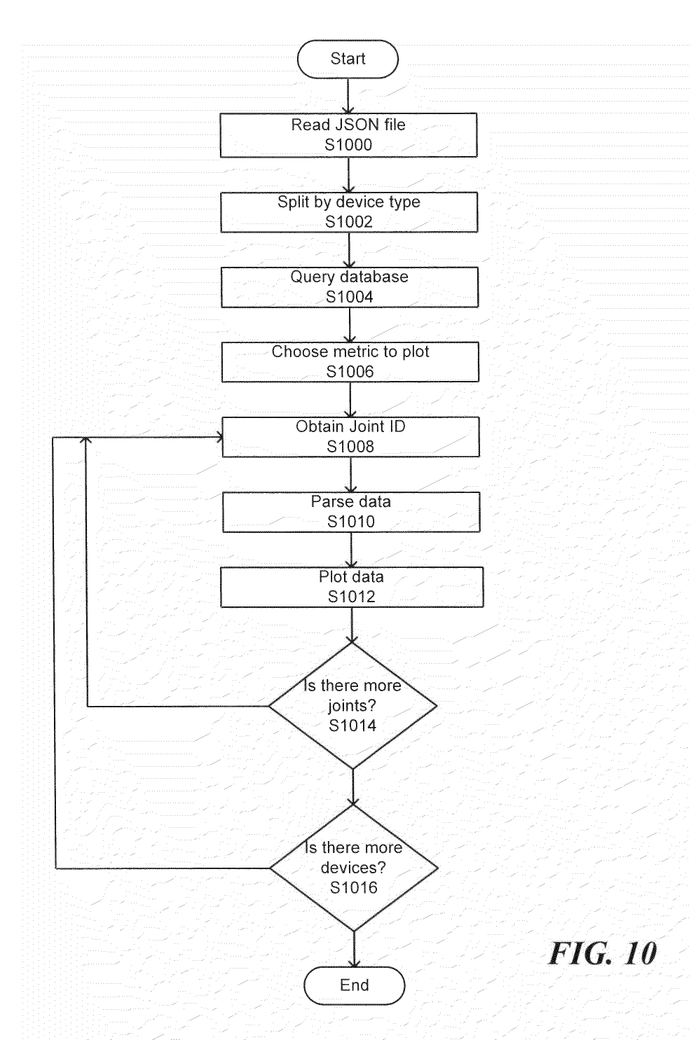

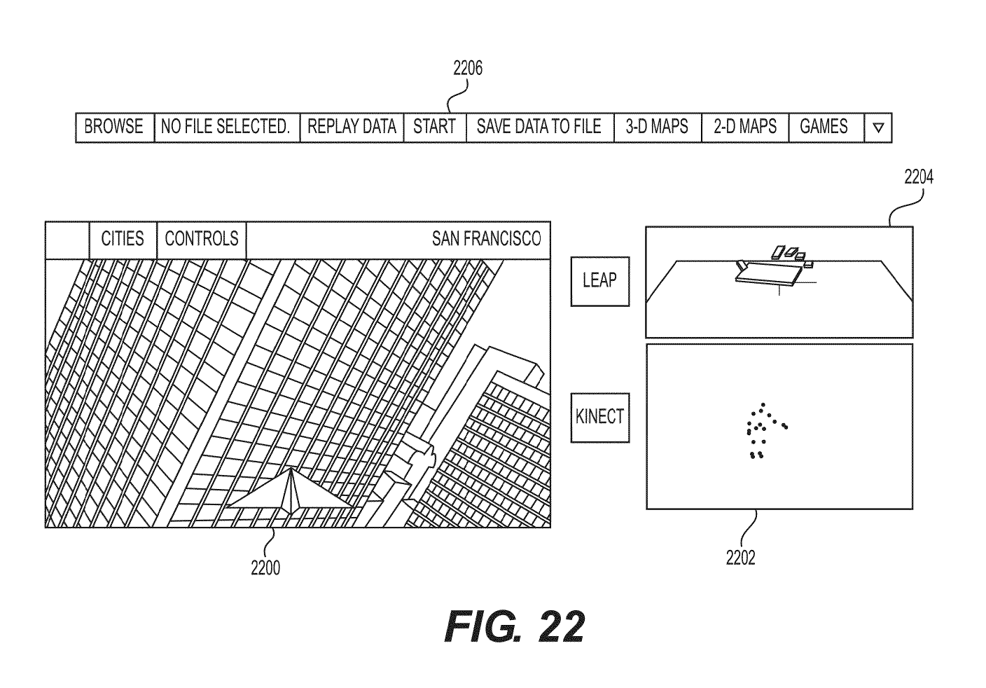

A reporting engine 712 takes the stored motion files and processes them to extract joint movement data. It converts the joint movement data to charts and plots them on the screen. For multiple joint movements, charts are plotted for each joint and its movement from top to bottom on the page, aligned by the time stamps as shown in FIG. 22. The charts may be used by the therapist to extract temporal information. The reporting engine 712 may use the method illustrated in FIG. 10. The reporting engine 712 feeds a reporting and visualization module 724. The reporting and visualization module 724 provides interactive graphs. The user may also plot past data retrieved from the health cloud 704. The system may also generate statistical progress report. The statistical progress report may be generated daily, weekly, monthly, yearly, or any other suitable period.

A GIS games repository 714 may store a plurality of GIS based serious game. A GIS game controller 716 may configure the game based on a patient rehabilitation status. A GIS game interface 720 is provided to the user as would be understood by one of ordinary skill in the art.

The software environment is set up such that a therapist can record an exercise session in 3D environment. The patient can log on to the framework and preview the hand therapy in 3D environment in the form of an avatar-hand on the screen. The system can record the patient's session and send it to the therapist. Temporal data collected from a number of sessions over a long period can be used to monitor the effectiveness and progress of the rehabilitation process.

In one embodiment, the system may display a video of the user performing the prescribed therapeutic exercises while the therapist is correcting him. This functionality provides high level of personalization and increases the accuracy of the patient performing the therapeutic exercises.

In selected embodiments, the server 100 based on the patient's current state and rate of improvement may select therapeutic exercises with a higher complexity and difficulty level.

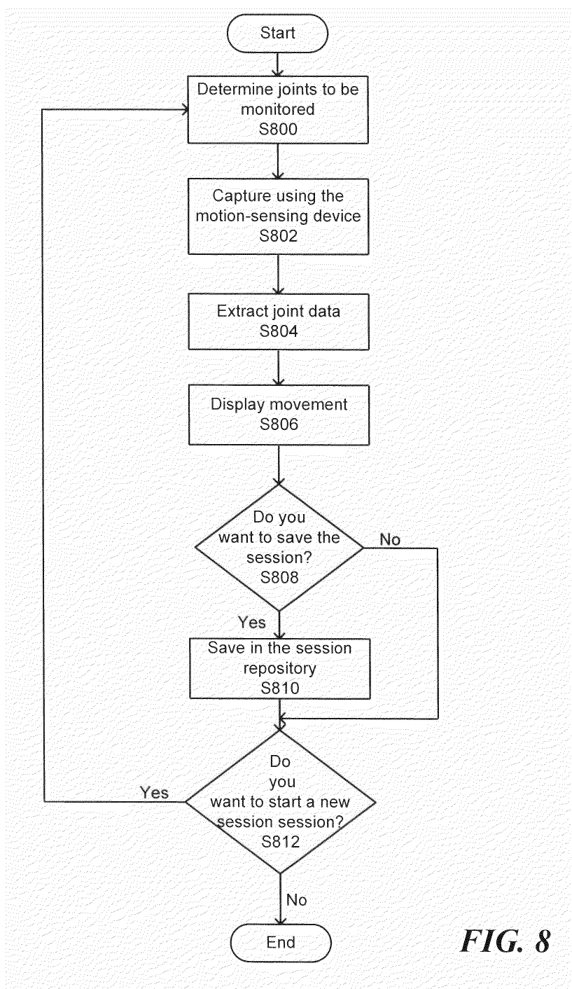

FIG. 8 is a flow chart to store a therapeutic session according to one example. At step S800, the user may choose the joints that need to be tracked during the therapeutic session. In one embodiment, the CPU 2400 may automatically determine which joints need to be tracked based on the user profile. At step S802, the selected joints movements are captured using the motion-sensing devices 104, 106. A joint movement may be captured by one or more motion-sensing devices. The data may be then fused to provide a higher accuracy. For example, a first motion-sensing device may have a higher lateral resolution than a second motion-sensing device while the second motion-sensing device may have a higher vertical resolution. The data collected from the two devices may be fused to provide a higher accuracy. Another example, one motion-sensing device may have a low resolution but a high acquisition rate. The data may be combined with a higher resolution device with a lower acquisition rate. The low resolution data may then be enhanced using the high resolution data. At step S804, the captured data is analyzed and the motion for the selected joints is extracted. At step S806, the metrics plots may be displayed to the user. At step S808, the user may choose to save or to disregard the session. In response to the user choosing to save the session, the CPU 2400 may save the session in the session repository 604, at step S810. At step S812, the user has the choice to start a new session. For example, the patient may choose to start another therapeutic sequence of exercises.

FIG. 9 is a flow chart for a motion analyzer according to one example. At step S900, a patient identification number is detected. The patient identification number may be detected by a plurality of ways. The patient identification number may be determined using a look-up table to match the fingerprint. The user may also enter the patient identification number by typing, by voice or by face recognition. The patient identification number may also be determined using the serial number of the motion-sensing device. At step S902, a therapy identification is detected. The therapy identification may be inputted by the patient, the caregiver or the medical professional. The therapy identification may be determined by the CPU 2400 through analyzing the user profile and prescribed therapy. The CPU 2400 may use the current time to determine the therapy identification. For example, in a patient user profile, “X00” may be performed every morning Once the CPU 2400 determines the current time, as in the morning, then the CPU 2400 may deduce that the therapy identification is “X00”. At S904, the data stream is read from the motion-sensing device. The CPU 2400 may automatically start reading the data stream once a motion is detected. At step S906, the CPU 2400 may determine the joints and the movements to be tracked. At step S908, the CPU 2400 may process the data stream to extract the data related to the joints and the movements to be tracked. The CPU 2400 may filter the data stream to remove data that falls out of a predetermined range. For example, if a joint movement has a known movement range from zero to thirty degrees. In response to the CPU 2400 determining that the data collected at an instance does not fall between zero and thirty degree, the data is discarded. At step S910, the QoI is updated. At S912, the CPU 2400 may check whether another frame is available. In response to determining that another frame is available, the flow goes to S908. At step S914, the CPU 2400 may check whether another joint need to be tracked. In response to determining that another joint needs to be tracked, the flow goes to S908. In response to determining that no other joint needs to be tracked, the flow goes to S916. At step S916, the CPU 2400 may check whether data stream from a secondary device is available. In response to determining that another device is available, the flow goes to step S904.

FIG. 10 is a flow chart that shows an algorithm used by the reporting engine according to one example. At step S1000, the CPU 2400 may read the data file. The data file may be in JSON format. At step S1002, the data stream is divided by device type. At step S1004, the CPU 2400 query the therapy database 608 to determine the set of joints corresponding to the therapy identification. At S1006, the metric to plot is determined. The user may choose what metric to plot. In one embodiment, the metric to plot is stored in the memory 2402. At step S1008, a joint identification number is determined. At step S1010, the data is parsed. At step S1012, the data is plotted. At step S1014, the CPU 2400 may check whether there are more joints to be tracked. In response to determining that there are more joints to be tracked, the flow goes to S1008. At step S1016, the CPU 2400 may check whether other devices are available. In response to determining, that other devices are available the flow goes to S1008.

In one embodiment, the server 100 using the CPU 2400 may generate an alert when the patient 108 did not perform the required therapeutic exercise. The server 100 may also generate the alert when the patient 108 does not complete the exercise. When the patient 108 skips the therapeutic exercise for more than a predetermined number, an alert is generated to the community of interest 112. For example, when a child skips the required therapeutic exercise or does not complete correctly an alert is generated to the parent or the guardian. For example, when a child performs the exercise less than a prescribed number, an alert may be generated.

In one embodiment, the CPU 2400 may compare the current state of a joint with a prescribed state stored in the therapy database. In response to determining that the joint state does not correspond with the prescribed state, the alert is generated. The alert may include a warning sound. The alert may also include generating an error message on the display. The error message may show the prescribed state.

In one embodiment, a reminder may be provided to the patient to perform the required therapeutic exercise. For example, the alert may be visual, audible or tactical. The alert may be shown on the patient's computer, television, smartphone, smartwatch, or the like.

The system may also poll a patient's electronic calendar to generate the reminder to perform the required therapeutic exercise at convenient times to the user. For example, the system may access an electronic calendar of the patient and the user profile. The system, using the CPU 2400, may determine a convenient time to perform the exercise. Then the system may generate an alert informing the user about the convenient time. For example, the therapist may indicate that the patient should perform the therapeutic session each morning. This information is stored in the user profile as explained above. The CPU 2400 may poll the electronic calendar of the patient to determine available free time during the morning. The CPU 2400 may then alert the patient to perform the therapeutic session at the available free time. In another example, the patient may perform its daily therapeutic session at 5 pm. The CPU 2400 may determine that the patient has an activity such as attending a birthday party at 5 pm and may then generate the alert to perform the therapeutic session at 4 pm. In this way, the system avoids constraints such as prayer times, school, or the like.

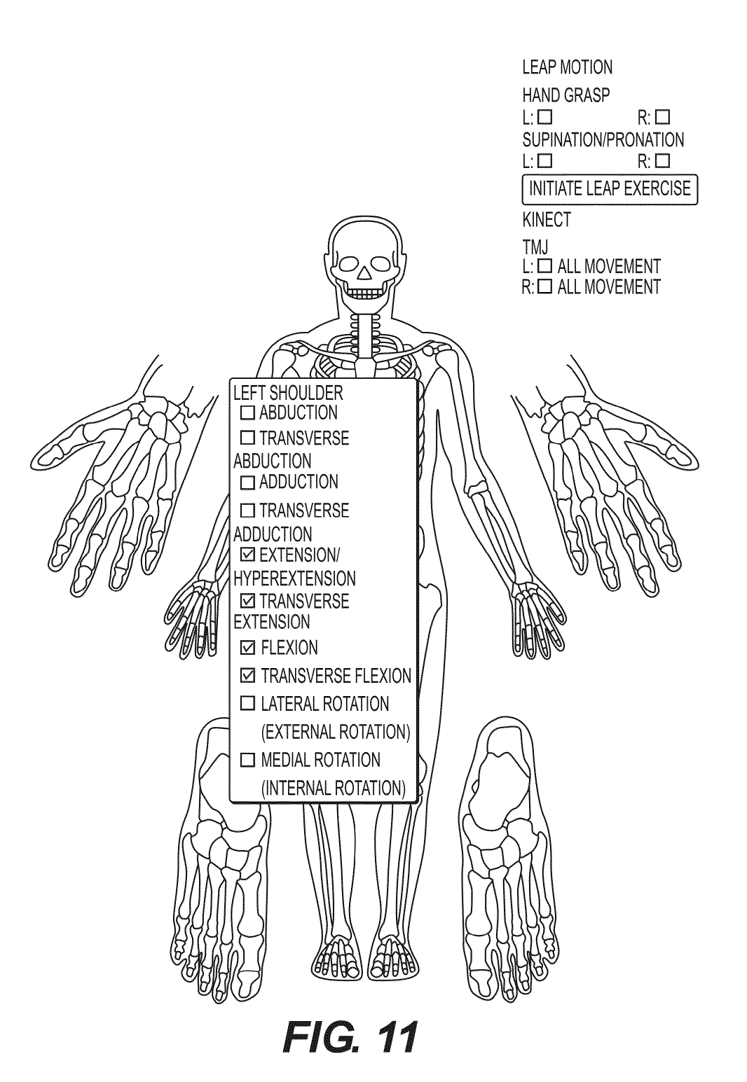

FIG. 11 is a user interface to select joints and their movements according to one example. FIG. 11 shows a human body anatomical model where each joint of the body is associated with a subset of therapeutic motions. In selected embodiments, the therapist may create the therapy by clicking on joints and selecting the movements shown in FIG. 11.

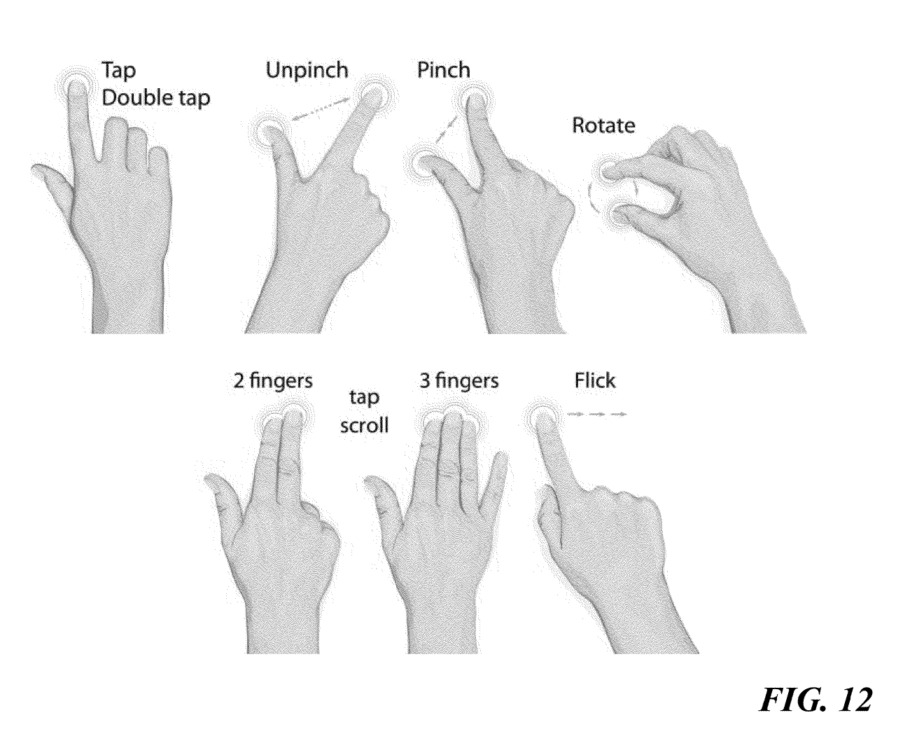

FIG. 12 is a schematic that shows a hand movement according to one example. Using the system of the present disclosure, the therapist can visualize live and statistical therapies incorporating complex motions that combine the primitive motions shown in FIGS. 4A, 4B and 5. FIG. 12 shows an exemplary complex and compound therapy imitating touch screen operation of a smartphone. The motions are then detected by the motion-sensing device. The range of motion is then determined and plotted by the framework.

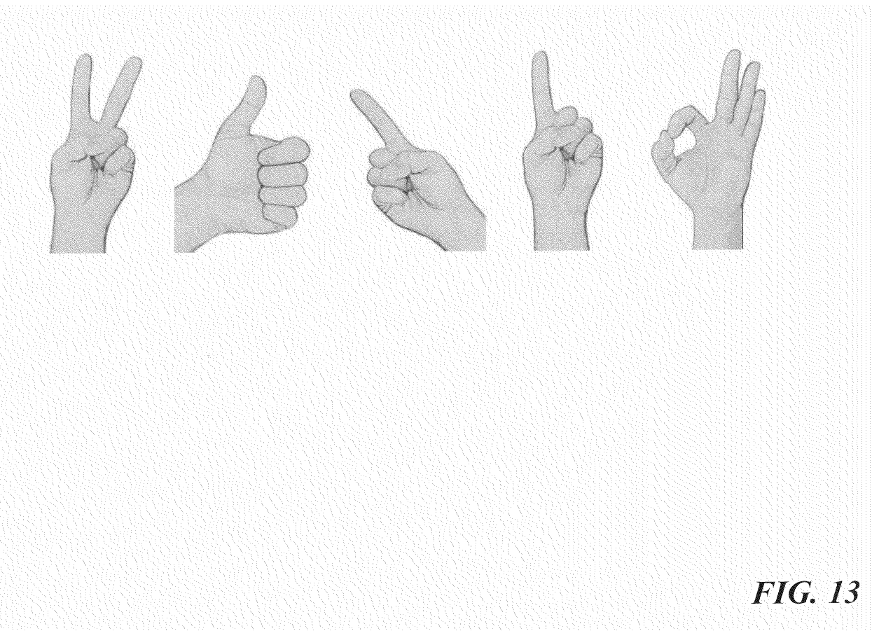

FIG. 13 is a schematic that shows complex and compound therapies according to one example. FIG. 13 shows an exemplary complex and compound therapy imitating American Sign Language (ASL). The motions are then detected by the motion-sensing device. The range of motion is then determined and plotted by the framework.

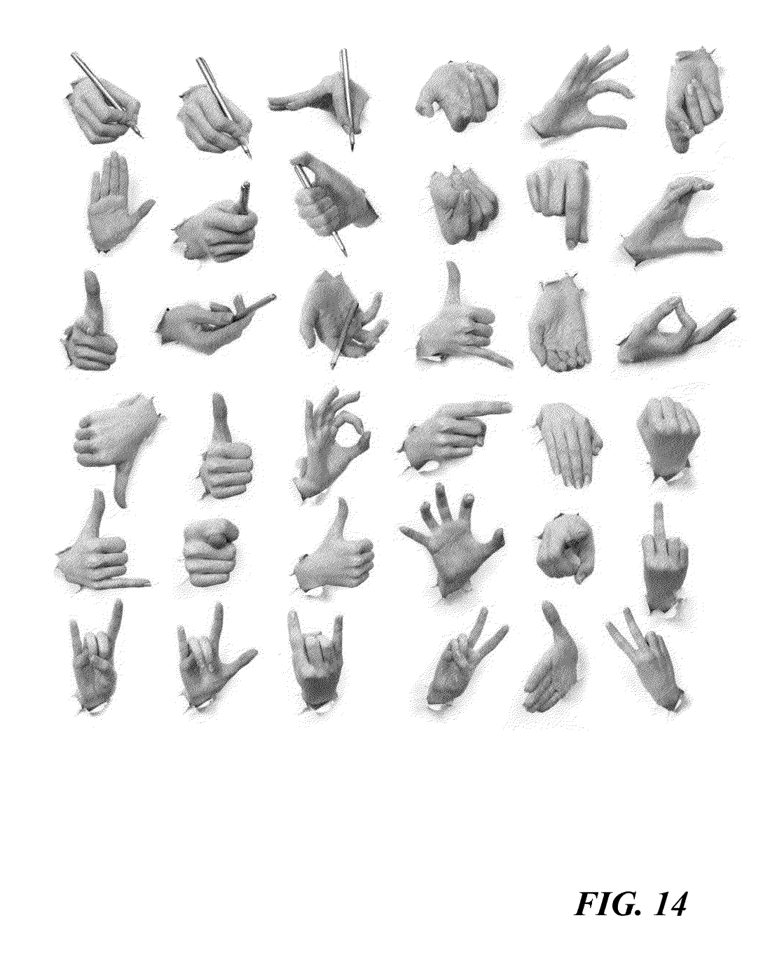

FIG. 14 is a schematic that shows complex and compound therapies according to one example. FIG. 14 shows therapies incorporating complex hand motions carrying objects such as a pen or actions requiring the combination of a plurality of the primitive motions. The complex and compound therapies may represent functional tasks such as writing. For each of the complex hand motions, the therapy database 608 may comprise a corresponding sequence of the primitive motions and a target ROM associated with each primitive motions.

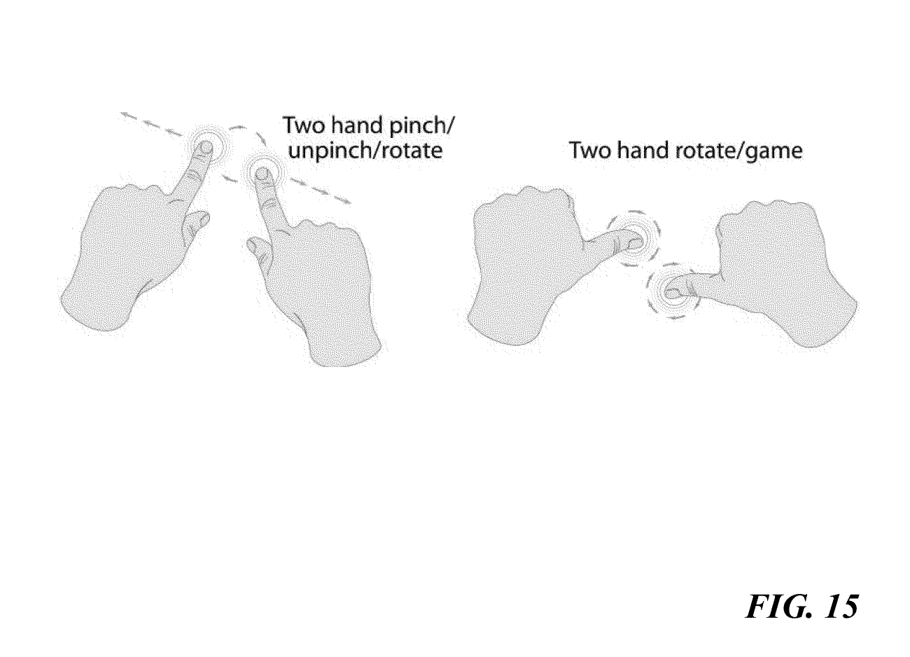

FIG. 15 is a schematic that shows complex and compound therapies according to one example. FIG. 15 shows a complex therapy that requires the use of both hands in the therapy environment. The movements of both hands may be detected by the motion-sensing device and analyzed by the system. The system using processing circuitry may show joint, finger and hand position in the 3D web-based environment in real time. Information metrics are extracted from the movement data. The information metrics may include speed of the movements, length of the bone connecting the joints, and a Range of Motion (ROM) of the primitive therapy discussed in FIGS. 4A, 4B and 5.

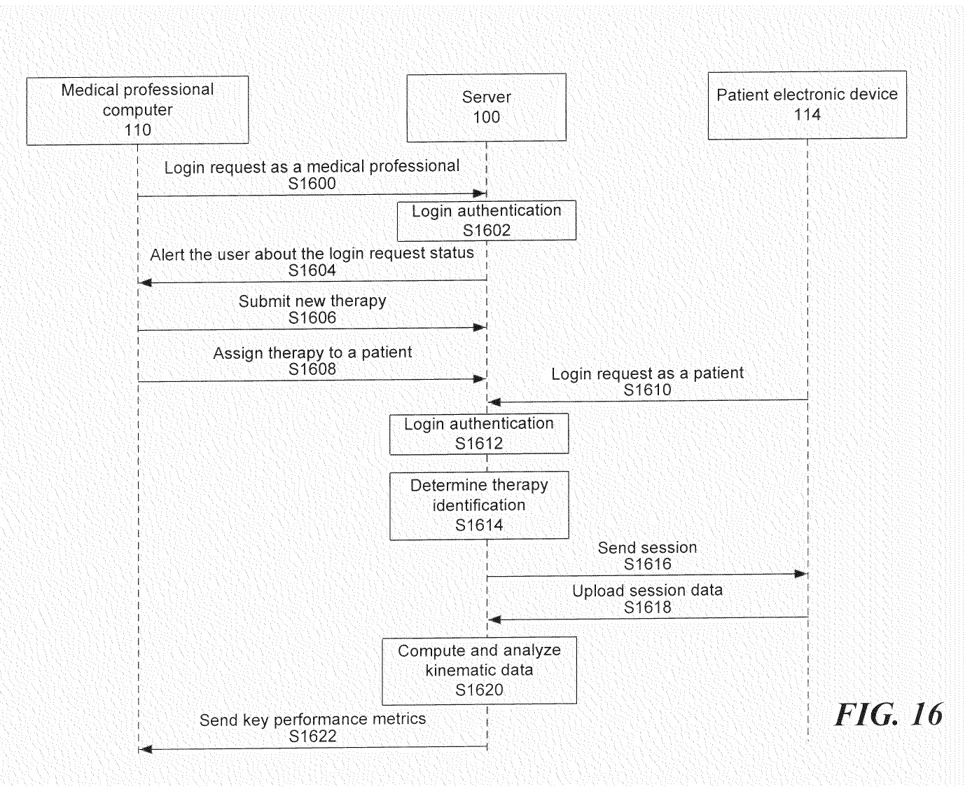

FIG. 16 is an exemplary flow chart that shows the operation of the system according to one example. At step S1600, the medical professional computer 110 may send a login request to the server 100. At step S1602, the server 100 validates the request. At step S1604, the server 100 alerts the user about the login request status. The server 100 may check whether the medical professional has the privilege to add new therapies. At step S1606, the medical professional may send a new therapy to the server. At step S1608, the medical professional using the medical professional computer 110 may assign the new therapy to one or more patients. At step S1610, the server 100 may receive a login request from a patient using the electronic device 114. At step S1612, the server 100 may authenticate the user. At step S1614, the server 100 using the CPU 2400 may determine the therapy identification corresponding to the patient. The CPU 2400 may use a look-up table that stores the patient identification number, the therapy identification, and a time when the therapy needs to be performed. At step S1616, the server 100 sends to the electronic device 114, the therapeutic exercise sequence. As described above, the therapeutic exercise sequence may be in a serious game format or as a video stream. At step S1618, the server 100 receives the session data stream. At step S1620, the server 100 using the CPU 2400, compute and extract kinematic data from the session data stream. At step S1622, the server 100 may send key performance metrics to the medical professional computer 110. The key performance metrics may include the range of motion. In selected embodiments, the server 100 may generate alerts to the medical professional when the patient is not doing the required number of therapy assigned to him. The alert may also be generated when the patient is not doing the therapeutic exercises in the right order. The server 100 may compute statistical analysis of performance during a certain time period such as a week, a month, a quarter or any suitable period.

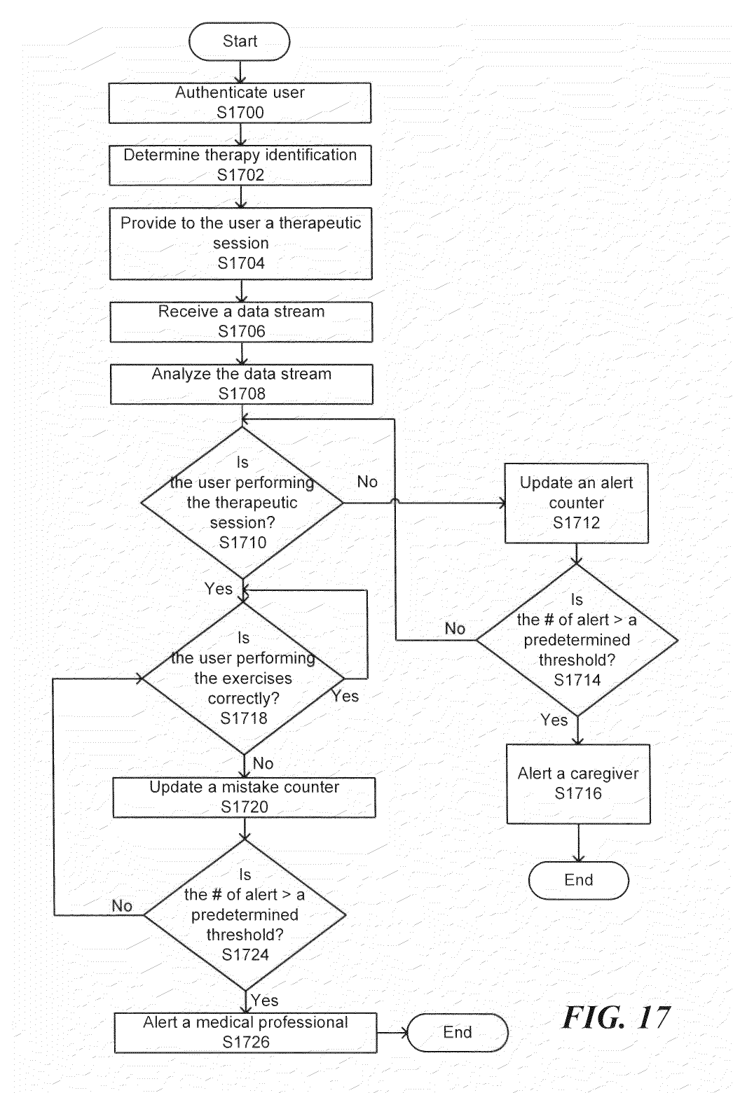

FIG. 17 is a flow chart that shows the operation of the system according to one example. At step S1700, the user is authenticated by any method as discussed above. At step S1702, the therapy identification is determined by using a look-up table to match the user identity with the therapy identification. At step S1704, the system may provide the user with the therapeutic session. At step S1706, the system may receive via the network 102, the data stream. At step S1708, the CPU 2400 may analyze the data stream to extract metrics as explained above. At step S1710, the CPU 2400 may determine whether the user is performing the therapeutic session. For example, the CPU 2400 may determine that the user is not performing the therapeutic session if the data stream contains only background noise. In response to determining that the user is not performing the therapeutic session, an alert count/number is increased by a predetermined incremental value. At step S1714, the CPU 2400 compares the alert number with a predetermined alert threshold. If the alert number is greater than the predetermined alert threshold then the alert to the caregiver is generated at S1716. The predetermined incremental value may be a function of the importance of the therapeutic session. For example, associated with each therapy identification may be an importance level such as “preventive”, “optional” or “required”. A therapy session with a higher importance level may have a higher predetermined incremental value when it is not performed by the user. At step S1718, the CPU 2400 may check whether the user is performing the exercises correctly. In response to determining that the user is not performing the exercise adequately, a mistake count/number is increased by a preset incremental value. The CPU 2400 may determine whether the user is performing the exercise correctly by comparing the user joints' state with the stored joint state of the corresponding exercise. The CPU 2400 may identify a mistake when the comparison is below a first threshold. At step S1724, the CPU 2400 compares the mistake count/number with a mistake threshold. If the mistake number is greater than the mistake threshold then an alert to the medical professional computer 110 is generated at step S1726 and transmitted to the caregiver via the network 102. In one embodiment, an alert counter is updated when the mistake count is above a second threshold. The CPU 2400 may generate an alert to the caregiver when the alert counter is greater than a third threshold.

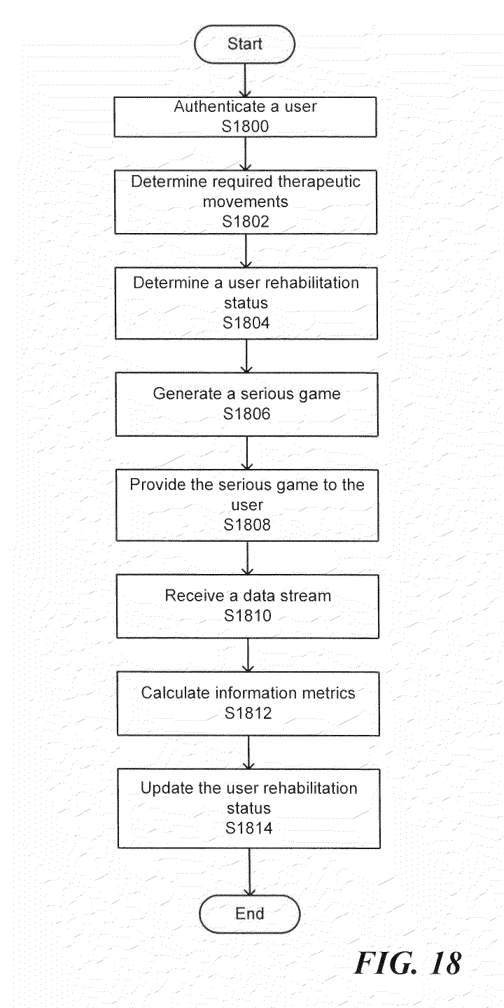

FIG. 18 is a flow chart that shows an algorithm to generate a serious game according to one example. At step S1800, the user is authenticated using any method described above. At step S1802, the CPU 2400 may determine required therapeutic movements by using the look-up table stored in the memory to match the user identity with the required therapeutic movements. At step S1804, a user rehabilitation status is determined based on the patient data stored in the memory 2402. The rehabilitation status may indicate current ROM of joints. At step S1806, the serious game is generated. As explained above the serious game may comprise browsing a map. The user performs the required therapeutic movements to browse the map. Each therapeutic movement is mapped to an action on the map (move up, move left, and the like). The ROM of each joint required while playing the game is based on the current ROM of joints of the user. At step S1808, the serious game is outputted via the network 102 on the display of the electronic device 114. At step S1810, the data stream form the motion-sensing device 104 is detected. At step S1812, the CPU 2400 may calculate the information metrics. At step S1810, the user rehabilitation status is updated based on the information metrics.

To illustrate the capabilities of the system, exemplary results are presented.

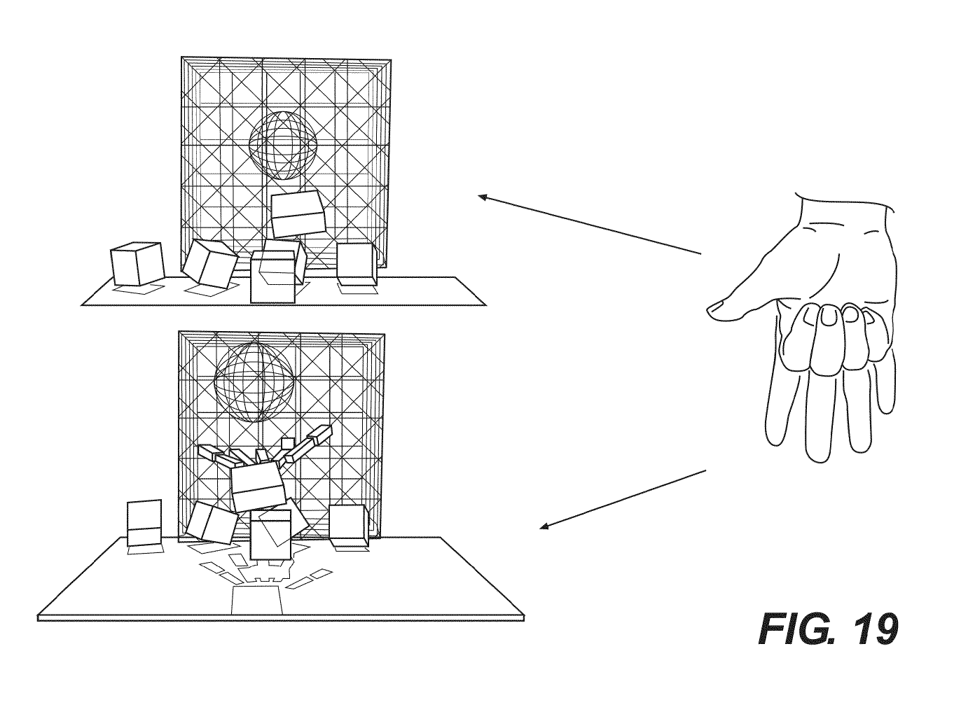

FIG. 19 is a schematic that shows an exemplary 3D live view of a therapeutic exercise according to one example. FIG. 19 shows live joint, finger and hand position in real time in the 3D web based environment. In one embodiment, the virtual representation of the hand may mimic the shape of a human hand. In other embodiments, the virtual representation of the hand may be an object, a dot, or the like. In one embodiment, the background scene may only comprise fixed elements. In other embodiments, the background scene may include moving objects. For example, the background scene may include a ball the patient is trying to catch.

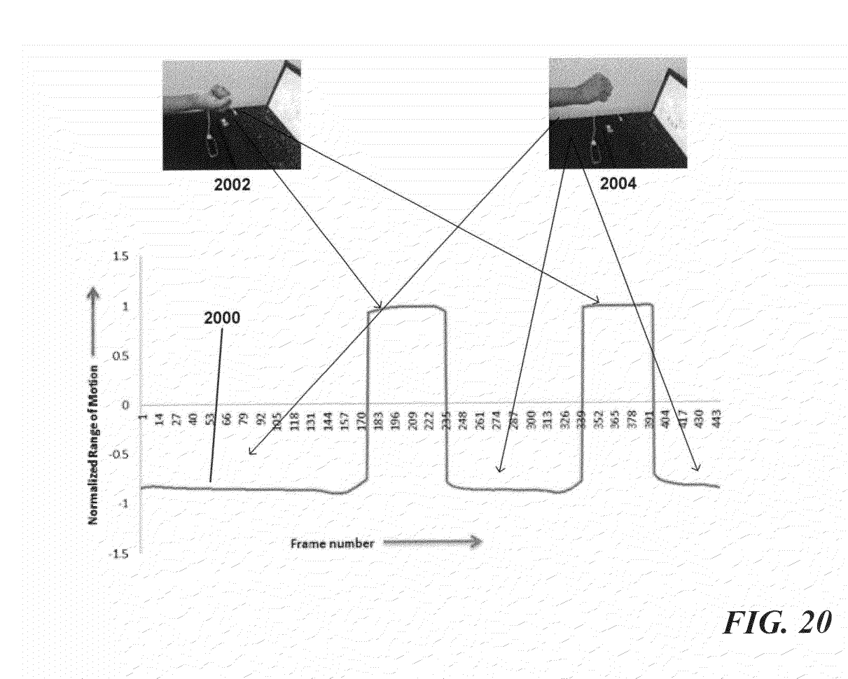

FIG. 20 is a schematic that shows the range of motion of a joint according to one example. The therapist may use the visualized kinematic motions and metrics and statistical analysis to clinically decide on the quality of improvement of the patient. Trace 2000 is an instantaneous plot showing normalized ROM of the hand. Schematic 2002 shows a patient hand in supination action. Schematic 2004 shows a patient hand in a pronation action. In trace 2000, the pronation action is indicated by negative y values and the supination action is indicated by positive values. In selected embodiments, the plots may be normalized to the full range of a healthy individual. In other embodiments, the trace may be normalized to the individual number. The trace may also show the patient's average, patient's best and patient's worst performance The trace may also show other patients' average. Results from a previous therapeutic session may also be plotted to the user who may see his progression.

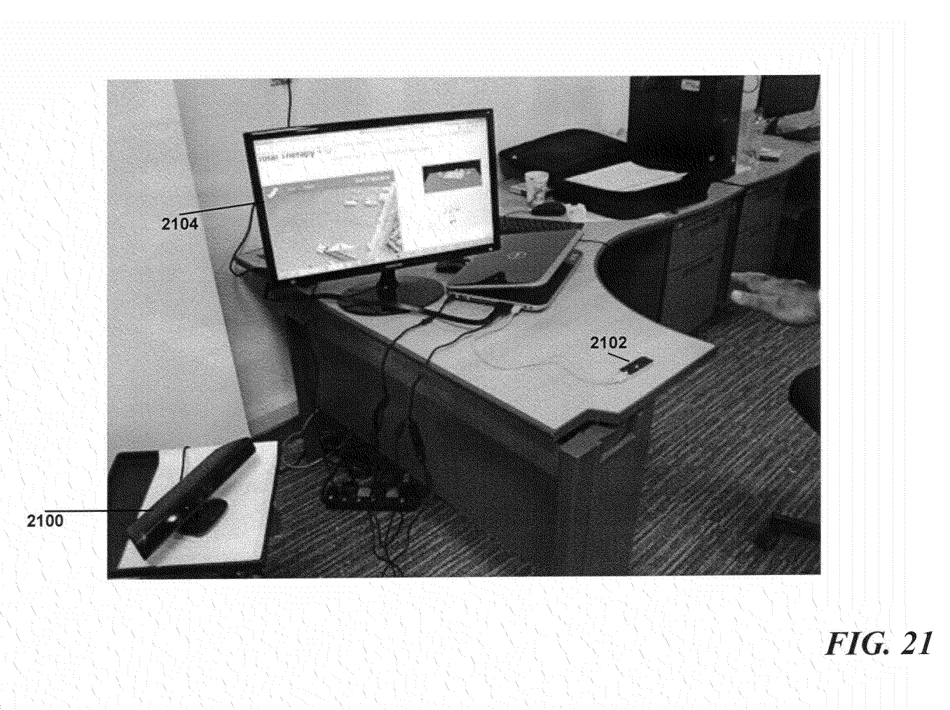

FIG. 21 is an image that shows an exemplary system setup according to one example. FIG. 21 shows two motion-sensing devices: the KINECT 2100 and the LEAP 2102. FIG. 21 shows a display 2104 that may be used to visualize the output interface.

FIG. 22 is an exemplary web based user interface according to one example. 2200 shows a web-based interface for a map browsing serious game application. 2204 shows a live 3D rendering of KINECT provided skeleton. 2204 shows a live rendering of LEAP stream showing hand skeleton. The user interface may also include a control menu 2206. The control menu 2206 may include buttons to save, select a file, start a session, pause a session or the like.

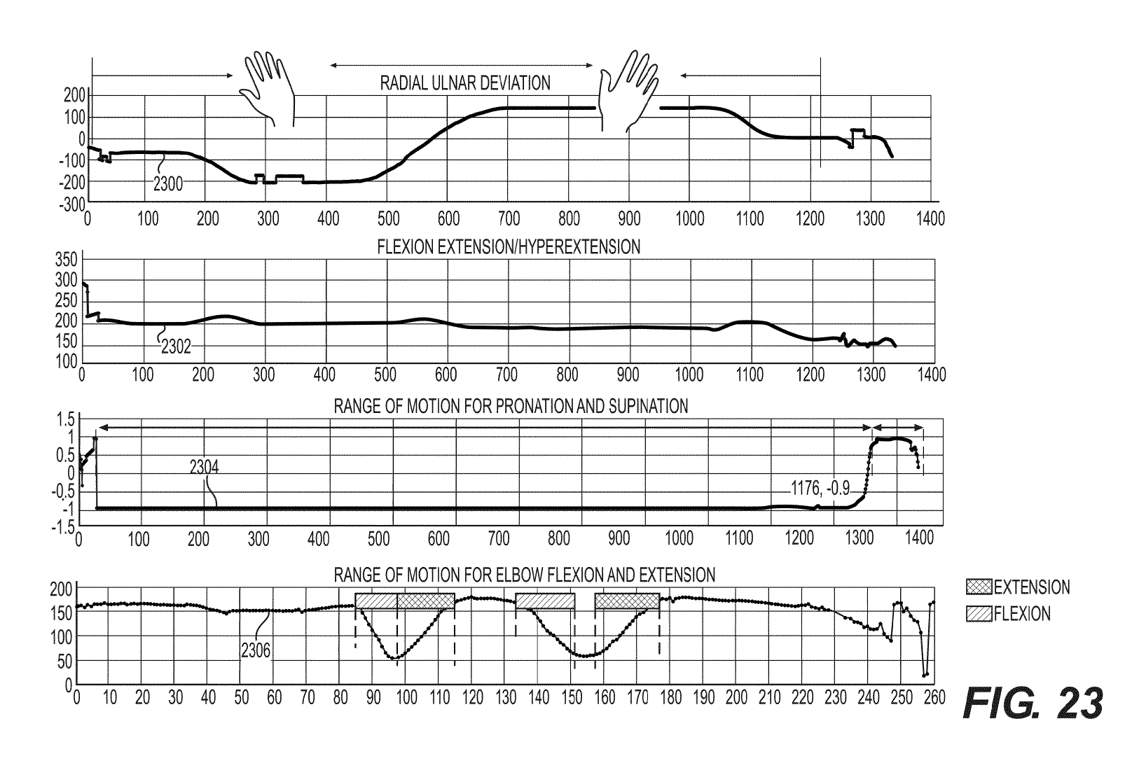

FIG. 23 is a chart that shows exemplary traces obtained from the inverse kinematic analyzer according to one example. A first trace 2300 shows a wrist radial-ulnar deviation. A second trace 2302 shows a flexion extension/hyperextension of the wrist. A third trace 2304 shows pronation-supination of palm surface. A fourth trace 2306 shows a flexion-extension of an elbow joint. In traces 2300, 2302, 2304, 2306 the x-axis represents the number of frames. The number of frames gives the temporal dimension. The CPU 2400 may determine movement speed, total therapy duration, time taken to complete one unit ulnar deviation or the like based on the number of frames. The y-axis shows a normalized range of motion. In the first trace 2300, the negative values show radial deviation or inclination of the thumb and fingers towards center of the body while the positive values show ulnar deviation or movement of fingers away from the center of the body. The first trace 2300 shows that the user moved his hand once in the right direction and one in the left direction. The second trace 2302 shows that the wrist was initially hyperextended for a short duration and then it was in the extension state for the rest of the therapy session. In the third trace 2304, the negative values show that the palm normal is facing downwards and hence the hand is in the state of pronation while the positive values show that the palm normal is facing upwards and so the hand is in a state of supination. In the fourth trace 2306, the falling curve represents a decrease in the distance between the wrist and the shoulder joint depicting flexion at the elbow while the rising curve (positive slope) shows an increase in the distance between the two thus representing extension at the elbow joint. The therapist or the caregiver may easily track the timeline and movements of the joints.

The patient may use a personal computer to connect the Kinect device through a USB port. The output interface may be displayed using a HTML5 based browser using a WebSocket.

Next, a hardware description of the server 100 according to exemplary embodiments is described with reference to FIG. 24. In FIG. 24, the server 100 includes a CPU 2400 which performs the processes described above/below. The process data and instructions may be stored in memory 2402. These processes and instructions may also be stored on a storage medium disk X04 such as a hard drive (HDD) or portable storage medium or may be stored remotely. Further, the claimed advancements are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the server 100 communicates, such as a server or computer.

Further, the claimed advancements may be provided as a utility application, background daemon, or component of an operating system, or combination thereof, executing in conjunction with CPU 2400 and an operating system such as Microsoft Windows 7, UNIX, Solaris, LINUX, Apple MAC-OS and other systems known to those skilled in the art.

The hardware elements in order to achieve the server 100 may be realized by various circuitry elements, known to those skilled in the art. For example, CPU 2400 may be a Xenon or Core processor from Intel of America or an Opteron processor from AMD of America, or may be other processor types that would be recognized by one of ordinary skill in the art. Alternatively, the CPU 2400 may be implemented on an FPGA, ASIC, PLD or using discrete logic circuits, as one of ordinary skill in the art would recognize. Further, CPU 2400 may be implemented as multiple processors cooperatively working in parallel to perform the instructions of the inventive processes described above.

The server 100 in FIG. 24 also includes a network controller 2406, such as an Intel Ethernet PRO network interface card from Intel Corporation of America, for interfacing with network 102. As can be appreciated, the network 102 can be a public network, such as the Internet, or a private network such as an LAN or WAN network, or any combination thereof and can also include PSTN or ISDN sub-networks. The network 102 can also be wired, such as an Ethernet network, or can be wireless such as a cellular network including EDGE, 3G and 4G wireless cellular systems. The wireless network can also be WiFi, Bluetooth, or any other wireless form of communication that is known.

The server 100 further includes a display controller 2408, such as a NVIDIA GeForce GTX or Quadro graphics adaptor from NVIDIA Corporation of America for interfacing with display 2410, such as a Hewlett Packard HPL2445w LCD monitor. A general purpose I/O interface 2412 interfaces with a keyboard and/or mouse 2414 as well as a touch screen panel 2416 on or separate from display 2410. General purpose I/O interface also connects to a variety of peripherals 2418 including printers and scanners, such as an OfficeJet or DeskJet from Hewlett Packard.

A sound controller 2420 is also provided in the server 100, such as Sound Blaster X-Fi Titanium from Creative, to interface with speakers/microphone 2422 thereby providing sounds and/or music.

The general purpose storage controller 2424 connects the storage medium disk 2404 with communication bus 2426, which may be an ISA, EISA, VESA, PCI, or similar, for interconnecting all of the components of the server 100. A description of the general features and functionality of the display 2410, keyboard and/or mouse 2414, as well as the display controller 2408, storage controller 2424, network controller 2406, sound controller 2420, and general purpose I/O interface 2412 is omitted herein for brevity as these features are known.

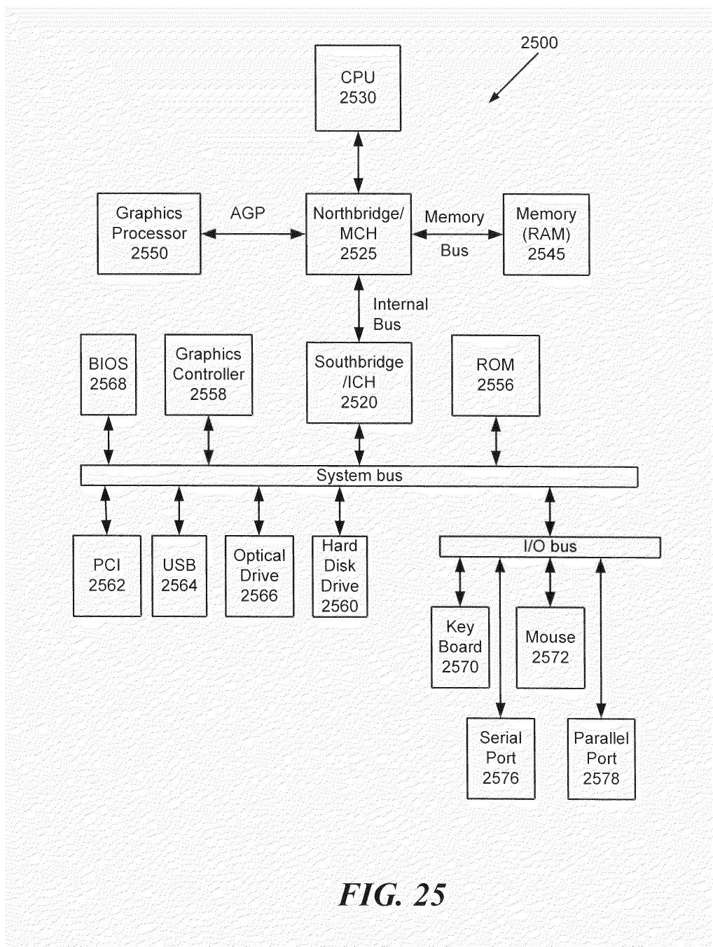

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 25.

FIG. 25 shows a schematic diagram of a data processing system, according to certain embodiments, for detecting, tracking and visualization of joint therapy data. The data processing system is an example of a computer in which code or instructions implementing the processes of the illustrative embodiments may be located.

In FIG. 25, data processing system 2500 employs a hub architecture including a north bridge and memory controller hub (NB/MCH) 2525 and a south bridge and input/output (I/O) controller hub (SB/ICH) 2520. The central processing unit (CPU) 2530 is connected to NB/MCH 2525. The NB/MCH 2525 also connects to the memory 2545 via a memory bus, and connects to the graphics processor 2550 via an accelerated graphics port (AGP). The NB/MCH 2525 also connects to the SB/ICH 2520 via an internal bus (e.g., a unified media interface or a direct media interface). The CPU Processing unit 2530 may contain one or more processors and even may be implemented using one or more heterogeneous processor systems.

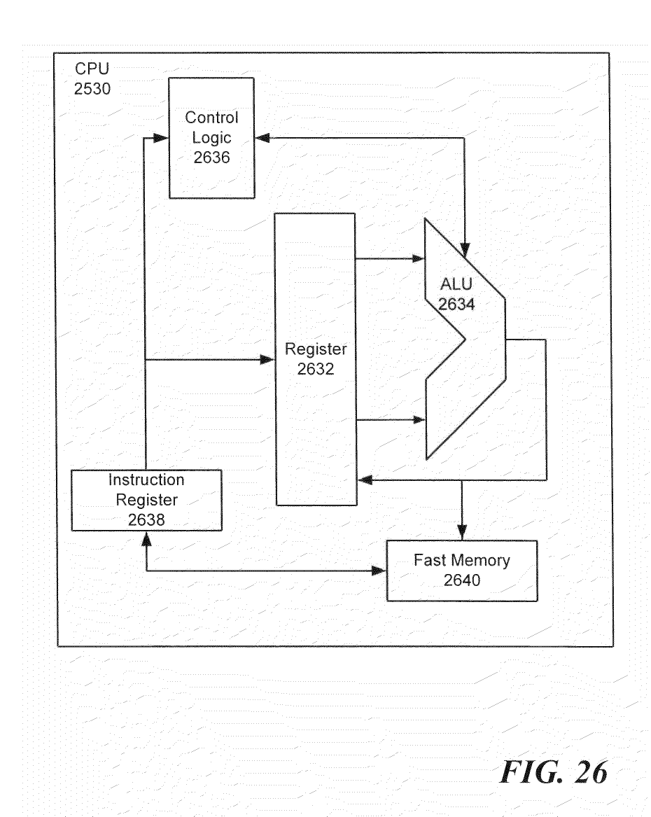

For example, FIG. 26 shows one implementation of CPU 2530. In one implementation, the instruction register 2638 retrieves instructions from the fast memory 2640. At least part of these instructions are fetched from the instruction register 2638 by the control logic 2636 and interpreted according to the instruction set architecture of the CPU 2530. Part of the instructions can also be directed to the register 2632. In one implementation, the instructions are decoded according to a hardwired method, and in another implementation, the instructions are decoded according a microprogram that translates instructions into sets of CPU configuration signals that are applied sequentially over multiple clock pulses. After fetching and decoding the instructions, the instructions are executed using the arithmetic logic unit (ALU) 2634 that loads values from the register 2632 and performs logical and mathematical operations on the loaded values according to the instructions. The results from these operations can be feedback into the register and/or stored in the fast memory 2640. According to certain implementations, the instruction set architecture of the CPU 2530 can use a reduced instruction set architecture, a complex instruction set architecture, a vector processor architecture, a very large instruction word architecture. Furthermore, the CPU 2530 can be based on the Von Neuman model or the Harvard model. The CPU 2530 can be a digital signal processor, an FPGA, an ASIC, a PLA, a PLD, or a CPLD. Further, the CPU 2530 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 25, the data processing system 2500 can include that the SB/ICH 2520 is coupled through a system bus to an I/O Bus, a read only memory (ROM) 2556, universal serial bus (USB) port 2564, a flash binary input/output system (BIOS) 2568, and a graphics controller 2558. PCI/PCIe devices can also be coupled to SB/ICH 2520 through a PCI bus 2562.

The PCI devices may include, for example, Ethernet adapters, add-in cards, and PC cards for notebook computers. The Hard disk drive 2560 and CD-ROM 2566 can use, for example, an integrated drive electronics (IDE) or serial advanced technology attachment (SATA) interface. In one implementation, the I/O bus can include a super I/O (SIO) device.

Further, the hard disk drive (HDD) 2560 and optical drive 2566 can also be coupled to the SB/ICH 2520 through a system bus. In one implementation, a keyboard 2570, a mouse 2572, a parallel port 2578, and a serial port 2576 can be connected to the system bust through the I/O bus. Other peripherals and devices that can be connected to the SB/ICH 2520 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

The hardware description above, exemplified by any one of the structure examples shown in FIG. 23, 24, or 25, constitutes or includes specialized corresponding structure that is programmed or configured to perform the algorithm shown in FIG. 16. For example, the algorithm shown in FIG. 16 may be completely performed by the circuitry included in the single device shown in FIG. 23 or the chipset as shown in FIG. 24.

The above-described hardware description is a non-limiting example of corresponding structure for performing the functionality described herein.

A system that includes the features in the foregoing description provides numerous advantages to users. In particular, the system helps to conduct the therapy in home and as many times as needed, and helps therapists viewing live therapy conducted in the patient's home. In addition, the system is easy to use, as it does not require any sensor to be attached to the human body.

Obviously, numerous modifications and variations are possible in light of the above teachings. It is therefore to be understood that within the scope of the appended claims, the invention may be practiced otherwise than as specifically described herein.

Thus, the foregoing discussion discloses and describes merely exemplary embodiments of the present invention. As will be understood by those skilled in the art, the present invention may be embodied in other specific forms without departing from the spirit or essential characteristics thereof. Accordingly, the disclosure of the present invention is intended to be illustrative, but not limiting of the scope of the invention, as well as other claims. The disclosure, including any readily discernible variants of the teachings herein, defines, in part, the scope of the foregoing claim terminology such that no inventive subject matter is dedicated to the public.

1. A game-based method for physical rehabilitation, the method comprising:
authenticating, via processing circuitry, a user with authentication information input through a communication interface;
identifying, using the processing circuitry, therapeutic movements by referencing a look-up table stored in a memory, the therapeutic movements being prescribed for the user or associated with a diagnosis of a predetermined physical condition;
obtaining, using the processing circuitry, a user rehabilitation status stored in the memory;
generating, using the processing circuitry, a game based on the therapeutic movements and the user status wherein the game includes controlling browsing of a map by detecting the therapeutic movements performed by the user;
providing, via communication circuitry, the game to the user;
receiving, via the communication circuitry, a data stream from a motion-sensing device that monitors an actual movement of the user in correspondence to the therapeutic movements;
analyzing, using the processing circuitry, the data stream to calculate information metrics that are associated with the correspondence of the actual movements to the therapeutic movements; and
updating the user rehabilitation status based on the information metrics.

2. The method of claim 1, wherein the game is displayed on a display in a 3D format.

3. The method of claim 1, wherein the map corresponds to a map of a city where the user resides, the city being included in the authentication information.

4. The method of claim 1, wherein the information metrics include at least one of a speed of movement, a range of motion, and a length of bone connecting joints.

5. The method of claim 1, wherein the game is a multiplayer game.

6. The method of claim 1, wherein the data stream is received from a plurality of motion-sensing devices.

7. The method of claim 1, wherein the processing circuitry selects a virtual background that corresponds with a user age.

8. The method of claim 1, further comprising:
calculating with the processing circuitry a rate of improvement of a user condition based on a state of the information metrics; and
generating an alert to a computer of a medical professional when the rate of improvement is less than a predetermined rate.

9. A game-based system for physical rehabilitation, the system comprising:
a motion-sensing device;
a memory;
communication circuitry; and
processing circuitry configured to
authenticate a user with authentication information input through a communication interface,
determine therapeutic movements by referencing a look-up table stored in the memory, the therapeutic movements being prescribed for the user or associated with a diagnosis of a predetermined physical condition,
obtain a user rehabilitation status stored in the memory,
generate a game based on the therapeutic movements and the user status wherein the game includes controlling browsing of a map by detecting the therapeutic movements performed by the user,
provide, via the communication circuitry, the game to the user,
receive, via the communication circuitry, a data stream from a motion-sensing device that monitors an actual movement of the user in correspondence to the therapeutic movements,
analyze the data stream to calculate information metrics that are associated with the correspondence of the actual movements to the therapeutic movements, and
update the user rehabilitation status based on the information metrics.

10. The game-based system of claim 9, wherein the game is displayed on a display in a 3D format.

11. The game-based system of claim 9, wherein the map corresponds to a map of a city where the user resides, the city being included in the authentication information.

12. The game-based system of claim 9, wherein the information metrics include at least one of a speed of movement, a range of motion, and a length of bone connecting joints.

13. The game-based system of claim 9, wherein the game is a multiplayer game.

14. The game-based system of claim 9, wherein the data stream is received from a plurality of motion-sensing devices.

15. The game-based system of claim 9, wherein the processing circuitry selects a virtual background that corresponds with a user age.

16. The game-based system of claim 9, wherein the processing circuitry is further configured to:
calculating with the processing circuitry a rate of improvement of a user condition based on a state of the information metrics; and
generating an alert to a computer of a medical professional when the rate of improvement is less than a predetermined rate.
